SEQUENCE LISTING (38 sequences)

SEQ ID NO: 1 - DNA, 354 bp, Artificial Sequence
  Synthetic hCASK-PDZopt
tttttatgcggcccagccggccggatccggtatggatatggaaaatgttacccgtgttcgtttagttcaatttcaaaaaaataccgatgaaccaatgggtattaccttgaaaatgaatgaattgaatcattgtattgttgcccgtattatgcatggtggtatgattcatcgtcaaggtactttgcatgttggtgatgaaattcgtgaaattaatggtatttctgttgccaatcaaaccgttgaacaattgcaaaaaatgttgcgtgaaatgcgtggttctattacctttaaaattgttccatcttatcgtacccaatcttcttctggaattcatgcggccgctggtgctccagt

SEQ ID NO: 2 - protein, 99 aa, Artificial Sequence
  Synthetic hCASK-PDZopt
GMDMENVTRVRLVQFQKNTDEPMGITLKMNELNHCIVARIMHGGMIHRQGTLHVGDEIREINGISVANQTVEQLQKMLREMRGSITFKIVPSYRTQSSS

SEQ ID NO: 3 - protein, 337 aa, Artificial Sequence
  hCASK-PDZ-GST fusion
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSRRASVGSGMDMENVTRVRLVQFQKNTDEPMGITLKMNELNHCIVARIMHGGMIHRQGTLHVGDEIREINGISVANQTVEQLQKMLREMRGSITFKIVPSYRTQSSSGIHRD

SEQ ID NO: 4 - protein, 590 aa, Artificial Sequence
  hCASK-PDZ-alkaline phosphatase  fusion
MSIQHFRVALIPFFAAFCLPVFAGMDMENVTRVRLVQFQKNTDEPMGITLKMNELNHCIVARIMHGGMIHRQGTLHVGDEIREINGISVANQTVEQLQKMLREMRGSITFKIVPSYRTQSSSRTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 5 - protein, 351 aa, Artificial Sequence [sequence omitted]

SEQ ID NO: 6 - protein, 128 aa, Artificial Sequence
  polyhistidine tagged and secreted hCASK-PDZ
MSIQHFRVALIPFFAAFCLPVFAGMDMENVTRVRLVQFQKNTDEPMGITLKMNELNHCIVARIMHGGMIHRQGTLHVGDEIREINGISVANQTVEQLQKMLREMRGSITFKIVPSYRTQSSSHHHHHH

SEQ ID NO: 7 - protein, 122 aa, Artificial Sequence
  secreted hCASK-PDZ
MSIQHFRVALIPFFAAFCLPVFAGMDMENVTRVRLVQFQKNTDEPMGITLKMNELNHCIVARIMHGGMIHRQGTLHVGDEIREINGISVANQTVEQLQKMLREMRGSITFKIVPSYRTQSSS

SEQ ID NO: 8 - protein, 234 aa, Artificial Sequence
  NHERF PDZ dimer
PRLCCLEKGPNGYGFHLHGEKGKLGQYIRLVEPGSPAEKAGLLAGDRLVEVNGENVEKETHQQVVSRIRAALNAVRLLVVDPETDEQLQKLGVQVREELLRAQEAPGQAEPPAAAEVQGAGNENEPREADKSHPEQRELRPRLCTMKKGPSGYGFNLHSDKSKPGQFIRSVDPDSPAEASGLRAQDRIVEVNGVCMEGKQHGDVVSAIRAGGDETKLLVVDRETDEFFKKCRVI

SEQ ID NO: 9 - protein, 546 aa, Artificial Sequence
  third PDZ domain of human Dlg1
MKKLLFAIPLVVPFYAAQPAAVLGDDEITREPRKVVLHRGSTGLGFNIVGGEDGEGIFISFILAGGPADLSGELRKGDRIISVNSVDLRAASHEQAAAALKNAGQAVTIVAQYRPEEYSRFEAAAAGAPVPYPDPLEPRAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 10 - DNA, 485 bp, Artificial Sequence
  third PDZ domain of human Dlg1
ttattattcgcaattcctttagttgttcctttctatgcggcccagccggccgcagtacttggagatgatgaaattacaagggaacctagaaaagttgttcttcatcgtggctcaacgggccttggtttcaacattgtaggaggagaagatggagaaggaatatttatttcctttatcttagccggaggacctgctgatctaagtggagagctcagaaaaggagatcgtattatatcggtaaacagtgttgacctcagagctgctagtcatgagcaggcagcagctgcattgaaaaatgctggccaggctgtcacaattgttgcacaatatcgacctgaagaatacagtcgttttgaagctgcggccgcaggtgcgccggtgccgtatccggatccgctggaaccgcgtgccgcatagactgttgaaagttgtttagcaaaacctcatacagaaaattcatttactaacgtctggaaagacgacaa

SEQ ID NO: 11 - protein, 9 aa, Artificial Sequence
  HA peptide
YPYDVPDYA

SEQ ID NO: 12 - protein, 11 aa, Artificial Sequence
  Tat peptide
YGRKKRRQRRR

SEQ ID NO: 13 - protein, 10 aa, Artificial Sequence
  hCASK ligand
QKAPTKEFYA

SEQ ID NO: 14 - DNA, 21 bp, Artificial Sequence
  pCAN5′ primer
catgattacgccaagctttgg

SEQ ID NO: 15 - DNA, 20 bp, Artificial Sequence
  pCAN3′ primer
cgatctaaagttttgtcgtc

SEQ ID NO: 16 - DNA, 59 bp, Artificial Sequence
  NNK1B  primer
  misc_feature 25, 26, 31, 32, 37, 38: n = a, c, g, or t
caatgattcaattcattcattttmnnggtmnnaccmnntggttcatcggtatttttttg

SEQ ID NO: 17 - DNA, 23 bp, Artificial Sequence
  NNK2A  primer
aaaatgaatgaattgaatcattg

SEQ ID NO: 18 - DNA, 59 bp, Artificial Sequence
  NNK2B  primer
  misc_feature 23, 24, 26, 27, 35, 36: n = a, c, g, or t
ggtaatagaaccacgcatttcmnnmnncattttmnncaattgttcaacggtttgattgg

SEQ ID NO: 19 - DNA, 21 bp, Artificial Sequence
  NNK3A  primer
gaaatgcgtggttctattacc

SEQ ID NO: 20 - DNA, 489 bp, Artificial Sequence
  Synthetically generated  hCASK PDZ peptide
  misc_feature 179, 180, 185, 186, 191, 192, 335, 336, 344, 345, 347,148: n = a, c, g, or t
catgattacgccaagctttggagccttttttttggagattttcaacgtgaaaaaattattattcgcaattcctttagttgttcctttctatgcggcccagccggccggatccggtatggatatggaaaatgttacccgtgttcgtttagttcaatttcaaaaaaataccgatgaaccannkggtnnkaccnnkaaaatgaatgaattgaatcattgtattgttgcccgtattatgcatggtggtatgattcatcgtcaaggtactttgcatgttggtgatgaaattcgtgaaattaatggtatttctgttgccaatcaaaccgttgaacaattgnnkaaaatgnnknnkgaaatgcgtggttctattacctttaaaattgttccatcttatcgtacccaatcttcttctggaattcatgcggccgcaggtgcgccggtgccgtatccggatccgctggaaccgcgtgccgcatagactgttgaaagttg

SEQ ID NO: 21 - DNA, 59 bp, Artificial Sequence
  TSM1B  primer
  misc_feature 26: n = a, c, g, or t
caatgattcaattcattcattttaanggtarbacccaktggttcatcggtatttttttg

SEQ ID NO: 22 - DNA, 23 bp, Artificial Sequence
  TSM2A  primer
aaaatgaatgaattgaatcattg

SEQ ID NO: 23 - DNA, 59 bp, Artificial Sequence
  TSM2B  primer
ggtaatagaaccacgcatttcmytmakcattttmwkcaattgttcaacggtttgattgg

SEQ ID NO: 24 - DNA, 21 bp, Artificial Sequence
  TSM3A  primer
gaaatgcgtggttctattacc

SEQ ID NO: 25 - DNA, 489 bp, Artificial Sequence
  Synthetically generated hCASK PDZ
  misc_feature 191: n = a, c, g, or t
catgattacgccaagctttggagccttttttttggagattttcaacgtgaaaaaattattattcgcaattcctttagttgttcctttctatgcggcccagccggccggatccggtatggatatggaaaatgttacccgtgttcgtttagttcaatttcaaaaaaataccgatgaaccamtgggtvytaccnttaaaatgaatgaattgaatcattgtattgttgcccgtattatgcatggtggtatgattcatcgtcaaggtactttgcatgttggtgatgaaattcgtgaaattaatggtatttctgttgccaatcaaaccgttgaacaattgmwkaaaatgmtkarkgaaatgcgtggttctattacctttaaaattgttccatcttatcgtacccaatcttcttctggaattcatgcggccgcaggtgcgccggtgccgtatccggatccgctggaaccgcgtgccgcatagactgttgaaagttg

SEQ ID NO: 26 - protein, 10 aa, Artificial Sequence
  C-terminus of Bc1A
SASIIIEKVA

SEQ ID NO: 27 - protein, 12 aa, Artificial Sequence
  Control peptide
HRRSARYLDTVL

SEQ ID NO: 28 - protein, 10 aa, Artificial Sequence
  peptide ligand of the third PDZ domain of human Dlg1
SSLQSLETSV

SEQ ID NO: 29 - protein, 84 aa, Xenopus leavis
RLVQFQKNTDEPMGITLKMNELNHCIVARIMHGGMIHRQGTLHVGDEIREINGISVANQTVEQLQKMLREMRGSITFKIVPSYR

SEQ ID NO: 30 - protein, 84 aa, Mus musculus; identical to SEQ ID NO: 29

SEQ ID NO: 31 - protein, 60 aa, Takifugu rubripes
REVAFEKNQSEPLGVTLKLNDKQRCSVARILHGGMIHRQGSLHEGDEIAEINGKSVANQT

SEQ ID NO: 32 - protein, 59 aa, Danio rerio
RMIGIRKKAGEPLGVTFRMDKGDLVIARILHGGLIDRQGLLHVGDIIKEVNGKDVGTNP

SEQ ID NO: 33 - protein, 84 aa, Mus musculus
KIIRLVKNSEPLGATIKKDEQTGAITVARIMRGGAADRSGLIHVGDELREVNGIPVEDKRPEEIIKILSQSKGAITFKIIPSTK

SEQ ID NO: 34 - protein, 80 aa, Anopheles gambiae str.
KMVGIRRNPDEPLGLTVEVDEHNQLVVARIIAGGMIDRQGLLHPGDVILEVNGVPVTTPEELQGEISVAKESVTLKIGPS

SEQ ID NO: 35 - protein, 24 aa, Lymnaea stagnalis
VDVLQSMLKEARGSIILKIVPSLR

SEQ ID NO: 36 - protein, 22 aa, Homo sapiens
VDQLQKAMKETKGMISLKVIPN

SEQ ID NO: 37 - protein, 22 aa, Artificial Sequence
  Synthetic construct
VDQLQKAMKETKGMISLKVIPN

SEQ ID NO: 38 - protein, 24 aa, Mus musculus
PEEIIKILSQSKGAITFKIIPSTK

(19) United States
(12) Patent Application Publication
(10) Pub. No.: US 2007/0218001 A1
(43) Pub. Date: Sep. 20, 2007

(54) Biotherapeutics, Diagnostics and Research Reagents

(75) Inventor: Simon Delagrave, Stoneham, MA (US)

Correspondence Address: FISH & RICHARDSON PC, P.O. BOX 1022, MINNEAPOLIS, MN, 55440-1022, US

(73) Assignee: BIOTECH STUDIO, LLC, CAMBRIDGE, MA (US)

(21) Appl. No.: 10/584,020

(22) PCT Filed: Jan. 4, 2005
(86) PCT No.: PCT/US05/00136

§ 371 Date: Nov. 8, 2006

Related U.S. Application Data
(60) Provisional application No. 60/534,214, filed on Jan. 5, 2004.
(60) Provisional application No. 60/554,102, filed on Mar. 18, 2004.

(51) Int. Cl. A61K 38/16; A61K 49/00; C07H 21/04; C07K 14/00; C07K 16/00; C12N 1/00; C12N 15/63; G01N 33/53

(52) U.S. Cl. 424/9.1; 435/243; 435/320.1; 514/12; 530/300; 530/350; 530/387.1; 536/23.1

(57) ABSTRACT

The present invention provides polypeptides that contain one or more PDZ domains and are useful in the detection of pathogens. The polypeptides of the invention are also useful in the diagnosis, treatment, and prevention of diseases. Also provided are methods of preparing polypeptides of the invention.

### FIELD OF THE INVENTION

The present invention relates to polypeptides that are useful in methods of detecting pathogens as well as diagnosing and treating diseases. The polypeptides contain at least one PDZ domain capable of binding with a target associated with a pathogen or disease state. In vitro evolution processes can be used to prepare the polypeptides of the invention.

### BACKGROUND OF THE INVENTION

Availability of proteins that specifically bind or interact with target proteins or other molecules has for some time been of importance in biology and medicine. For example, medical diagnosis has been revolutionized by assays using high-affinity proteins, mainly antibodies, that bind to disease markers. High-affinity antibodies to disease-causing agents are of increasing importance in medical therapeutics. In biological research, high affinity proteins, also mainly antibodies, have found use in the purification of rare proteins, in the localization of proteins or other antigens in cells such as by immuno-histochemical techniques, and in countless other applications. High-affinity proteins are likely to assume increasing research importance in the future. For example, the emerging field of proteomics seeks to understand the patterns of expression and interaction of a substantial fraction of the proteins encoded in a cell's genome.

However, existing methods of providing binding proteins or polypeptides that bind with affinity and specificity to selected targets, especially to large numbers of selected targets, has been and continues to be difficult and expensive. The predominant method used today is to raise antibodies, either monoclonal or polyclonal, against a target molecule. Although well known and widely used, this strategy has several limitations and disadvantages. First, to generate, or “raise”, an antibody against a target requires either a sufficient amount of the purified target itself or a chemically synthesized fragment of the target. Second, raising an antibody normally requires the use of living animals, and due to species incompatibilities, it is not always possible to raise a specific antibody against a particular target, much less against large numbers of targets, such as a significant fraction of the proteins in an organism. Third, isolation and production of antibodies are expensive, time-consuming and unpredictable processes. Fourth, antibodies cannot be expressed via recombinant hosts without significant investment of time and money because the antigen-binding regions of the antibody heavy and light chains must be cloned, sequenced, and then simultaneously expressed. Finally, antibodies usually do not fold properly in the reductive cell environment, and therefore are not useful to target intracellular molecules involved in disease. Such limitations and disadvantages constitute a significant barrier to the rapid identification, diagnosis and treatment of infectious diseases such as AIDS, SARS, West Nile virus, and anthrax, or of non-infectious diseases such as cancer.

An alternative method relies on “directed evolution” to alter the binding specificity of naturally-occurring proteins that are known to bind to determined targets. In this method, a known gene is randomly mutated by a chemical or biotechnological mutagenesis technique, for example, by PCR-based mutagenesis. Then a library of the resulting protein variants is screened for variants having affinity to a new target, for example, by phage display. In this way, several proteins have been “evolved” in the laboratory to create protein variants having useful new specificities (e.g., Xu et al., 2002, Chem Biol, 9: 933).

A further alternative is to create novel binding proteins de novo through directed evolution. However, proteins having no natural counterparts, e.g., iMabs from Catchmabs BV or as described by (Keefe et al., 2001, Nature, 410: 715-8), have a significant drawback in that they are likely to be recognized as foreign by the human immune system, thereby impeding their use as therapeutics. For the same reason, natural proteins of non-human origin engineered to bind target polypeptides (e.g., Ronnmark et al., 2002, Eur J Biochem, 269: 2647-55.; Zeytun et al., 2003, Nat Biotechnol, 21: 1473-9.) are unlikely to be useful as therapeutics or diagnostics.

Thus, the choice of binding protein to be modified via directed evolution will strongly influence the utility of the evolved binding proteins. PDZ domains constitute an example of a family of binding proteins which can be used to create novel research reagents, diagnostic reagents or therapeutics having many advantages over existing binding proteins. Such advantages include ease and speed of isolation using in vitro methods, low cost of production using non-mammalian host cells, potential utility as intracellular biotherapeutics due to their natural propensity to function in the cytoplasm, and lack of immunogenicity.

PDZ domains are relatively well understood and of great potential utility. They participate in signal transduction pathways by mediating protein complex formation and are also involved in targeting of proteins to various locations within the cell. In metazoan genomes, including the human genome, PDZ domains are among the most common protein sequence modules. Recent reviews on PDZ domains include refs. (Hung et al., 2002, J Biol Chem, 277: 5699-702) and (Fan et al., 2002, Neurosignals, 11: 315-21). Many PDZ domains are stable and expressed to high levels in recombinant bacterial hosts, which has facilitated their extensive biophysical characterization (e.g., Morais Cabral et al., 1996, Nature, 382: 649-52.; Cohen et al., 1998, J Cell Biol, 142: 129-38.; Daniels et al., 1998, Nat Struct Biol, 5: 317-25.; Im et al., 2003, J Biol Chem, 278: 8501-7). PDZ domains have been described as potential therapeutics, for example to treat cancer by interfering with Myc protein function. See for example, (Junqueira et al., 2003, Oncogene, 22: 2772-81) and US patent application 20030119716. Other PDZ patent applications expand the utility of PDZ domains by describing engineered PDZ domain fusions, or chimeras, with other proteins (e.g., US Patent Application Pub. Nos. 20010044135, 20020037999, 20020160424). PDZ domains can also be used to identify drug candidates in high-throughput screens (Ferrer et al., 2002, Anal Biochem, 301: 207-16; Hamilton et al., 2003, Protein Sci, 12: 458-67).

Some progress has been made in studying and modifying the binding specificity of PDZ domains. Schneider et al., 1999, Nature Biotechnology 17:170-175 and (Junqueira et al., 2003, Oncogene, 22: 2772-81) both describe how the binding specificity of a naturally-occurring PDZ domain can be altered using directed evolution methods. Phage display may be used to determine the specificity of a given PDZ domain (see, e.g., Full et al., 2000, J. Biol. Chem. 275:21486-91). In this work, Full and colleagues selected phage-displayed random C-terminal peptide sequences capable of binding to an immobilized PDZ domain. However, this approach is not intended to, and cannot alter the specificity of a given PDZ domain. In contrast, Skelton et al. (2003, J. Biol. Chem., 278: 7645-54), propose the use of phage display to alter PDZ domain specificity, but do not demonstrate it. Phage display is believed to provide greater control over the conditions of the binding interactions, including affinity and specificity, than is afforded by two-hybrid selections which are notoriously artifact-prone.

Alternatively, PDZ domains with altered binding specificity may be designed by computational methods, as shown by (Reina et al., 2002, Nat Struct Biol, 9: 621-7) and US Patent Application Pub. No. 20030059827. These computational methods seem to offer several apparent benefits, such as reduced cost and time by avoiding experimental effort, and scalability for determining binding proteins to multiple targets. On the other hand, these methods have certain notable drawbacks such as the well-known extreme difficulty of predicting binding affinities of designed protein structures, yielding candidate binding proteins of unreliable affinity and specificity. Also, once structures have been designed in silico, the corresponding proteins must still be prepared in the laboratory. The effort required to construct the candidate gene variants is similar to the effort required to prepare a library of mutant genes, and once such a library is constructed, it can be screened multiple times with diverse targets whereas new variants must be designed and synthesized for each new target. Finally, design of variant binding proteins and optimization of their binding affinity is extremely difficult without the availability of detailed information on their atomic structure, while directed evolution has no such need. The acquisition of this type structural data is costly and slow, often requiring months of work.

In summary, polypeptides capable of binding to specific targets, especially to natural peptide sequences, are useful in biology and medicine, and are expected to be of increasing utility in the future. But the current art offers no methods sufficiently efficient and economical to meet demands for large numbers of versatile binding proteins. Existing methods are time consuming, often costly, and may have additional drawbacks. Therefore, inexpensive and efficient methods for providing diverse binding proteins capable of functioning as affinity reagents and/or therapeutics are needed.

### SUMMARY OF THE INVENTION

The present invention provides a polypeptide comprising an engineered PDZ domain, wherein said engineered PDZ domain binds to a target associated with a pathogen or disease state. In some embodiments, the pathogen is viral, fungal, or bacterial. In some embodiments, the pathogen is of the genus Bacillus. In some embodiments, the pathogen is Bacillus anthracis or Clostridium botulinum. In some embodiments, the disease state is cancer. In some embodiments, the target is a polypeptide. In some embodiments, the target is found in the exosporium of Bacillus anthracis. In some embodiments, the target is protein BclA of Bacillus anthracis or a fragment thereof. In some embodiments, the target is a polypeptide having a C-terminal sequence of EFYA. In some embodiments, the target is selected from IgA, IgD, IgM, IgG, IgE, interleukin, cytokine, amyloid beta, beta 2-microglobulin, VEGF, F protein of RSV, VP1 of Coxsackievirus A9, Vpr of HIV, PSA, and growth hormone. In some embodiments, the PDZ domain is evolved. In some embodiments, the evolved PDZ domain binds to a target with a dissociation constant (K_{d}) of about 100 nM or lower, about 50 nM or lower, about 20 nM or lower or about 15 nM or lower. In some embodiments, the PDZ domain is evolved from the PDZ domain of protein hCASK. In some embodiments, the PDZ domain is evolved from SEQ ID NO: 2. In some embodiments, the PDZ domain is a variant of the PDZ domain of protein hCASK. In some embodiments, the PDZ domain is a variant of SEQ ID NO: 2. In some embodiments, the PDZ domain is evolved from the third PDZ domain of human Dlg1. In some embodiments, the PDZ domain is evolved from SEQ ID NO: 9 or fragment thereof. In some embodiments, the PDZ domain is a variant of the third PDZ domain of human Dlg1. In some embodiments, the PDZ domain is a variant of SEQ ID NO: 9 or fragment thereof.

In some embodiments, the polypeptide of the invention further comprises a reporter group such as an enzyme, fluorescent protein, or epitope.

In some embodiments, the polypeptide of the invention further comprises an effector domain such as an antibody fragment, toxin, polyethylene glycol (PEG) moiety, or protein transduction domain.

In some embodiments, the polypeptide of the invention further comprises a radioactive isotope.

In some embodiments, the polypeptide is isolated.

The present invention further provides a polynucleotide encoding a polypeptide of the invention, a vector comprising the polynucleotide, a host cell comprising the polynucleotide, or an antibody that binds to the polypeptide of the invention. In some embodiments, the polynucleotide, vector, host cell or antibody is isolated.

The present invention further provides a method of detecting the presence of a pathogen or disease in a patient comprising:

a) administering a polypeptide of the invention to said patient; and

b) detecting binding of said polypeptide in said patient.

The present invention further provides a method of detecting the presence of a pathogen or disease in a sample comprising:

a) contacting a polypeptide of the invention (optionally comprising a reporter group) with said sample; and

b) detecting binding of said polypeptide to said sample.

In some embodiments, the detecting is carried out by Western blot or ELISA. In some embodiments, the sample comprises a bacterial pathogen. In some embodiments, the sample comprises Bacillus anthracis, Clostridium botulinum or their toxins. In some embodiments, the sample comprises a viral pathogen.

The present invention further provides a method of preparing a polypeptide comprising a PDZ domain, wherein said PDZ domain binds to a target produced by a pathogen or disease state, comprising:

a) creating a library of polypeptides from one or more parent polypeptides comprising a ADZ domain;

b) identifying one or more polypeptides from said library having binding affinity for said target.

In some embodiments, the library of polypeptides is created by combinatorial mutagenesis. In some embodiments, the library of polypeptides is created by error-prone PCR. In some embodiments, the one or more parent polypeptides is optimized for expression in a desired expression system. In some embodiments, the expression system is bacterial or yeast. In some embodiments, the identifying is carried out in a cell-free screening assay. In some embodiments, the identifying is carried out by phage display.

The present invention further provides a polypeptide comprising a PDZ domain and an effector domain. In some embodiments, the effector domain comprises a protein transduction domain, an Fc domain, or serum albumin.

The present invention further provides a polypeptide comprising a PDZ domain, wherein said PDZ domain binds to a target, wherein said polypeptide is prepared by:

a) creating a library of polypeptides from a parent polypeptide comprising a PDZ domain having SEQ ID NO: 2 or the sequence of the third PDZ domain of human Dlg1;

b) identifying said polypeptide having binding affinity for said target from said library.

In some embodiments, the target is associated with a pathogen or disease. In some embodiments, the said disease is cancer. In some embodiments, the pathogen is bacterial.

The present invention further provides a polypeptide comprising a PDZ domain, wherein said PDZ domain binds to a target, wherein said polypeptide is prepared by recursive ensemble mutagenesis. In some embodiments, the target is associated with a pathogen or disease state. In some embodiments, the disease is cancer. In some embodiments, the pathogen is bacterial.

The present invention further provides a library of polypeptides prepared from a parent polypeptide comprising a PDZ domain, said parent polypeptide comprising SEQ ID NO: 2 or the third PDZ domain of human Dlg1.

The present invention further provides a method of treating a disease, comprising administering to a patient afflicted with or likely to become afflicted with said disease a therapeutically effective amount of a polypeptide comprising a PDZ domain capable of binding to a target associated with the disease.

The present invention further provides a method of treating a disease associated with a pathogen, comprising administering to a patient infected with or likely to become infected with said pathogen a therapeutically effective amount of a polypeptide comprising a PDZ domain capable of binding to a target associated with said pathogen. In some embodiments, the pathogen is Bacillus anthracis. In some embodiments, the target comprises a toxin produced by Bacillus anthracis. In some embodiments, the Clostridium botulinum. In some embodiments, the target comprises a toxin produced by Clostridium botulinum. In some embodiments, the pathogen is Clostridium tetani. In some embodiments, the target comprises a toxin produced by Clostridium tetani.

The present invention further provides a method of preparing a polypeptide comprising a PDZ domain, wherein said PDZ domain binds to a polypeptide target associated with a pathogen, comprising:

a) forming a library of polypeptides from one or more parent polypeptides comprising a PDZ domain;

b) selecting a first polypeptide from said library, said first polypeptide having binding affinity to an intermediate target having 20% to 80% sequence identity in the last 5 amino acids with the last 5 amino acids of said target;

c) creating a further library of polypeptides from the first polypeptide of step b);

d) repeating steps b) and c) until a polypeptide that binds with said target is identified.

The present invention further provides a method of purifying a protein comprising contacting said protein with an immobilized polypeptide comprising a PDZ domain, wherein said immobilized polypeptide has binding affinity for said protein.

The present invention further provides a polypeptide comprising a PDZ domain, wherein said PDZ domain binds to a target with a dissociation constant (K_{d}) of 15 nM or lower, or 2 nM or lower. In some embodiments, the PDZ domain is evolved.

The present invention further provides a method of preparing a polypeptide comprising two or more PDZ domains, wherein said two or more PDZ domains bind to one or more targets, comprising:

a) creating a library of polypeptides from a parent polypeptide comprising two or more PDZ domains;

b) identifying one or more polypeptides from said library having binding affinity for said one or more targets.

The present invention further provides a polypeptide comprising two or more PDZ domains, wherein said PDZ domains bind to one or more targets, and wherein at least one of said PDZ domains is evolved. In some embodiments, the at least one of said PDZ domains binds to a target produced by a pathogen or disease state.

### BRIEF DESCRIPTION OF THE DRAWINGS

FIG. 1. Multiple sequence alignment of proteins found in a BLAST search of the “nr” protein database using hCASK PDZ domain as a query. Identities are shown as dots. Upper panel of figure shows relevant sequences for residues M501, I503 and L505. Lower panel shows relevant sequences for residues Q553, L556 and R557. Underlined numbers on left-hand side of the figure correspond to NCBI GI numbers.

### DESCRIPTION OF EMBODIMENTS OF THE INVENTION

The present invention provides, inter alia, a polypeptide comprising one or more PDZ domains capable of binding to a preselected target, and methods of using and preparing the polypeptide. In some embodiments, the PDZ domain is engineered. In some embodiments, at least one of the one or more PDZ domains binds to a target produced by a pathogen or disease state. In further embodiments, the polypeptide comprises two PDZ domains, advantageously resulting in a PDZ dimer that binds to a target peptide with greater avidity than a polypeptide containing only one PDZ domain. Example targets include naturally and non-naturally occurring proteins, peptides, or other molecules associated with, such as are associated with (e.g., produced directly or indirectly by), a pathogen or disease state, including non-infections disease states such as cancer, neurodegeneration, and cardiopulmonary dysfunction.

Definitions

As used herein, “polypeptides” or “proteins” are polymers of amino acids having, for example, from 2 to about 1000 or more amino acid residues. In some embodiments, “polypeptides” have from 10 to about 250 amino acids, or from about 15 about 200 amino acids. Any naturally occurring or synthetic amino acid can form the polypeptide. Polypeptides can also include modifications such as glycosylations and other moieties. In some embodiments, polypeptides of the invention have the ability to selectively bind to target polypeptides based on, for example, amino acid sequence of the target, such as amino acid sequences of the N- or C-terminus. Polypeptides of the invention contain at least one PDZ binding domain. In some embodiments, polypeptides can contain additional functional regions such as a “reporter group” and/or an “effector domain.”

As used herein, “engineered” refers to a polypeptide of the invention containing at least one PDZ domain that has been modified by in vitro manipulation. For example, an “engineered” polypeptide or PDZ domain is non-naturally occurring, such as a PDZ domain whose properties, including sequence, have been changed by in vitro mutation according to any suitable method including rational design or directed evolution. The engineered polypeptide typically has properties that differ from a naturally occurring polypeptide, such as different binding specificity or affinity. An “engineered” PDZ domain includes an “evolved” PDZ domain that has been subject to directed evolution or other in vitro evolution techniques.

As used herein, “PDZ domain” refers to a protein module capable of binding to a target protein by recognition of the target's C-terminal or N-terminal amino acid sequence. PDZ domains are typically 85-95 amino acids in length and are found naturally in a variety of organisms ranging from bacteria to humans. An example PDZ domain is the PDZ domain of hCASK having the sequence SEQ ID NO: 2. A further example PDZ domain is the third PDZ domain of human Dlg1, such as shown within SEQ ID NO:9 (see Example 16). Other PDZ domains, according to the invention, have homology to the PDZ domain of SEQ ID NO: 2 or SEQ ID NO: 9, such as at least about 50% identity using BLAST (default parameters). The name PDZ is derived from: PSD-95 (Cho et al., Neuron 9:929-942, 1992), Dlg-A (Woods and Bryant, Cell 66:451-464, 1991) and ZO-1 (Itoh et al., J. Cell. Biol. 121:491-502, 1993), each of which contains three such domains. PDZ domains have also been called GLGF repeats or DHRs and are identified in a variety of proteins (Ponting and Phillips, Trends Biochem. Sci. 20:102-103, 1995). A PDZ domain of PTPL1 has been shown to interact with the C-terminal tail of the membrane receptor Fas (Sato et al., 1995) and PDZ domains of PSD-95 bind to the C-terminals of the NMDA-receptor and Shaker-type K⁺channels (Kim et al., Nature 378:85-88, 1995; Kornau et al., Science 269:1737-1740, 1995). The crystal structures of different PDZ domains have been published (e.g., Doyle et al., Cell 0.85:1067-1076, 1996; Morais Cabral et al., Nature 382:649-652, 1996). The PDZ domain of human CASK/LIN-2, also called hCASK, is well studied: its substrate specificity has been investigated (Cohen et al., 1998, J Cell Biol, 142: 129-38.) and its crystal structure determined (Daniels et al., 1998, Nat Struct Biol, 5: 317-25.). One skilled in the art can readily recognize and identify a PDZ domain, for example, by using the CD-Search computer program available at www.ncbi.nlm.gov/Structure/cdd/cdd.shtml, the NIH's free “Conserved Domain Database and Search Service”.

PDZ domains can also be changed by an in vitro evolution process to generate an evolved PDZ domain having a particular desired function that is different from the original function. The “evolved” PDZ domain can be evolved from any parent PDZ domain, such as a naturally-occurring PDZ domain, to change binding affinity or specificity of the parent PDZ domain for a preselected target. In some embodiments, the evolved PDZ domain is evolved from the hCASK PDZ domain. In some embodiments, the evolved PDZ domain is evolved from the third PDZ domain of human Dlg1, the structure of which is discussed in Cabral et al., Nature 382:649-652, 1996.

A “reporter group,” as used herein, is defined as a molecular moiety that is readily detected, directly or indirectly, and is attached covalently to a polypeptide, such as a polypeptide of the invention containing a PDZ domain. Examples of reporter groups include polynucleotides that are readily detected, for example, by polymerase chain reaction (PCR); biotin which is readily detected with streptavidin conjugated to horseradish peroxidase; fluorescent proteins such as the Green Fluorescent Protein (GFP), which is detected by fluorescence spectroscopy; epitope tags such as the influenza hemagglutinin peptide HA epitope corresponding to the amino acid sequence YPYDVPDYA (SEQ ID NO: 11), detected with antibodies binding specifically to this epitope; dual function epitope/enzyme tags such as GST (glutathione S-transferase), which can be detected indirectly using an antibody specific to this protein, or directly using a colorimetric assay measuring enzymatic GST activity; enzymes such as alkaline phosphatase, which can be detected using chemiluminescence. Reporter groups also include radioactive isotopes and imaging agents, such as chelated heavy metals, which can be used for in vivo diagnostics and imaging. Numerous other examples of molecular entities which can be used as reporter groups are known in the art.

An “effector domain”, as used herein, is defined as a protein domain, or other molecular moiety, which adds a function other than detection to a polypeptide to which it is covalently attached. An effector domain can be the Fc domain of immunoglobulins, which mediates function of the immune system such as opsonization, phagocytosis and activation of complement. Other effector domains include toxins such as cholera toxin, which can be used to kill cells recognized by the polypeptide to which the effector domain is attached. Other toxins can include, for example, botulin toxin, diphtheria toxin, anthrax toxin, ricin, Clostridium difficile toxin, and the like. Other examples of effector domains include protein transduction domains which enable proteins to which they are attached to cross the cell membrane and to locate in the cytoplasm of mammalian cells, as described, for example, in Wadia and Dowdy, 2002, Curr Op Biotechnol, 13:52-6 and references therein, in which short sequences such as the Tat protein's transduction domain (YGRKKRRQRRR (SEQ ID NO: 12) single letter amino acid code) and other arginine-rich basic peptides are described. Another example effector domain is serum albumin. Yet other examples of effector domains include RNA molecules which can be used to mediate selective inactivation of gene expression via RNA interference (RNAi); chemotherapeutic agents such as bleomycin, which can be used to kill cancer cells; radioactive isotopes which can also be used to kill cancer cells; and the like. More than one effector domain can be linked to a single PDZ domain in a polypeptide of the invention. Effector domains such as PDZ domains binding to serum proteins or other host proteins can modulate pharmacokinetics of the protein to which it is fused. Thus, a PDZ domain having therapeutic activity can be fused to another PDZ domain acting as an effector domain modulating pharmacokinetics. Other molecules, such as polyethylene glycol (PEG), can also be used as effector domains to modulate pharmacokinetics or reduce immunogenicity (Nucci et al., Advan. Drug Del. Rev. 6, 133, 1991, and Inada et al., J. Bioactive Compat. Polymer 5,343, 1990). PEG can be attached to other proteins as described in U.S. Pat. No. 6,677,438.

As used herein, the term “variant” is meant to indicate a polypeptide differing from another polypeptide by one or more amino acid substitutions resulting from engineered mutations in the gene coding for the polypeptide. One skilled in the art can readily recognize and identify a variant of a PDZ domain, for example, by using the CD-Search computer program available at www.ncbi.nilm.gov/Structure/cdd/cdd.shtml, the NIH's free “Conserved Domain Database and Search Service” which can identify protein domains such as the PDZ domain and its variants. A polypeptide is typically no longer considered a variant of a parent polypeptide when the degree of homology between these polypeptides falls below about 40%, as ascertained for example by using the program BLAST to align two sequences (default parameters) described by Tatiana A. Tatusova and Thomas L. Madden (1999), “Blast 2 sequences—a new tool for comparing protein and nucleotide sequences”, FEMS Microbiol Lett. 174:247-250. In some embodiments, variants have at least about 50%, at least about 60%, at least about 70%, at least about 80%, at least about 85%, at least about 90%, at least about 95%, at least about 97%, at least about 98%, at least about 99% homology with the parent polypeptide, as ascertained for example by using the program BLAST to align two sequences (default parameters). In some embodiments, a parent polypeptide can be evolved in vitro using directed evolution to yield one or more variants of the parent polypeptide. These variants can have new or improved properties compared to the parent polypeptide or be useful in generating further variants.

The term “peptide” refers to a compound of 2 to about 50 subunit amino acids, amino acid analogs, or peptidomimetics. The subunits can be linked by peptide bonds. In other embodiments, the subunit can be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. In some embodiments, peptides can have from 2 to about 30, 2 to about 20, 2 to about 10, 9, 8, 7, 6, 5, 4, 3 or 2 subunit amino acids, amino acid analogs, or peptidomimetics.

As used herein, the term “in vitro evolution”, or “directed evolution” refers to a method of generating two or more different polypeptides (e.g., a “library” of polypeptides) by accelerating mutation rates and/or recombination events of polynucleotides encoding parent polypeptides under in vitro conditions and screening or selecting the resulting new polypeptides. The process of directed evolution has been described in detail (Joo et al., Chem. Biol., 1999, 6, 699-706; Joo et al., Nature, 1999, 399, 670-673; Miyazaki et al., J. Mol. Evol., 1999, 49, 716-720; Chen et al., Proc. Natl. Acad. Sci. USA, 1993, 90, 5618-5622; Chen et al., Biotechnology, 1991, 9, 1073-1077; You et al., Protein Eng, 1996, 9, 77-83; each of which is incorporated herein by reference in its entirety). In general, the method involves the steps of 1) creating a population of mutant polynucleotides; 2) screening this population for individual nucleotides which have a desired property such as coding for a protein with improved binding affinity; and repeating these two steps, if necessary, until a desired improvement is achieved. Many methods to introduce mutations exist and are described in the literature (Leung et al., Technique, 1989, 1, 11-15; Delagrave et al., Protein Eng., 1993, 6, 327-331; each of which is incorporated herein by reference in its entirety). Similarly, there are many ways to screen or select mutants for a desired property (Smith, Science, 1985, 228, 1315; Hanes & Pluckthun, Proc. Natl. Acad. Sci. USA, 1997, 94, 4937; Xu et al., Chem. Biol, 2002, 9, 933; Joo et al., Chem. Biol., 1999, 6, 699-706; Joo et al., Nature, 1999, 399, 670-673; Miyazaki et al., J. Mol. Evol., 1999, 49, 716-720; Chen et al., Proc. Natl. Acad. Sci. USA, 1993, 90, 5618-5622; Chen et al., Biotechnology, 1991, 9, 1073-1077; You et al., Protein Eng, 1996, 9, 77-83; Marrs et al., Curr. Opin. Microbiol., 1999, 2, 241-245; and U.S. Pat. No. 5,914,245).

As used herein, the term “parent polypeptide” describes a polypeptide which is a starting component of an in vitro evolution process. “Parent polypeptide” distinguishes the starting polypeptides from evolved forms of the polypeptides (“evolved polypeptides”). For example, a “parent PDZ domain” refers to a PDZ domain that is used as a starting point for generating different (or evolved) PDZ domains by in vitro evolution. Likewise, an “evolved PDZ domain” describes a PDZ domain that is the product of an in vitro evolution process. A parent PDZ domain can be a naturally-occurring domain.

As used herein, the term “parent polynucleotide” describes a polynucleotide which is a starting component of an in vitro evolution process. “Parent polynucleotide” distinguishes the starting polynucleotides from evolved forms of the polynucleotides (“evolved polynucleotides”). For example, a “parent PDZ polynucleotide” refers to a polynucleotide encoding a PDZ domain that is used as a starting point for generating different (e.g., evolved) PDZ domains (variant PDZ domains) by in vitro evolution. Likewise, an “evolved PDZ polynucleotide” describes a polynucleotide encoding a PDZ domain which is the product of an in vitro evolution process.

As used herein, “library” refers to a collection of two or more different polypeptides or polynucleotides. The collection of polypeptides or polynucleotides of a library can be prepared by any of numerous methods including error-prone PCR, recursive ensemble mutagenesis, combinatorial mutagenesis, and other mutagenesis methods such as gene shuffling and the like.

As used herein, “disease state” or “disease” or “disorder,” used interchangeably, refers to any of numerous pathological conditions of the mind or body. The disease state can be infectious or non-infectious. The disease state can be symptomatic or non-symptomatic infection by a pathogen. The disease state can be chronic or acute, and also includes abnormal immune responses (e.g., allergies). Example disease states include pathogen infection or toxicity due to exposure to pathogen-related toxins such as bacterial (e.g., botulism, anthrax), fungal, or viral infection. Further, example disease states include non-infectious diseases such as cancers (prostate, breast, etc.), cardiopulmonary diseases (myocardial infarction, atherosclerosis, etc.), neurodegenerative diseases (Alzheimer's, Parkinson's, ALS, etc.), allergic responses (e.g., asthma, hives, etc.) and the like.

As used herein, a “target” refers to any molecular entity to which a further molecular entity binds. In some embodiments, the target is a polypeptide or peptide. In further embodiments, at least one terminus, such as the C-terminus, is at least partially exposed. The target can be associated with a biological state such as a disease (pathogenic or non-pathogenic) or disorder in a plant or animal (e.g., a mammal) as well as the presence of a pathogen. When a target is “associated with” a certain biological state, the presence or absence of target or the presence of a certain amount of target (e.g., outside of normal levels), can identify the biological state. For example, a target can be a protein, such as prostate-specific antigen (PSA), that is differentially expressed in certain cancer cells. In some embodiments, the target can be amyloid beta (involved in Alzheimer's disease) or beta 2-microglobulin (involved in dialysis-associated amyloidosis) or peptides corresponding to the C-terminal 3 to 12 residues of these polypeptides. As a further example, a target can include proteins such as IgE (immunoglobulin E), IL-5, or IL-17, associated with diseases such as asthma. As a further example, a target can include proteins such as IgA, IgD, IgM, IgG. In further embodiments, the target can be interleukin, cytokine, amyloid beta, beta 2-microglobulin, VEGF, F protein of RSV, VP1 of Coxsackievirus A9, Vpr of HIV, PSA, or growth hormone.

As a further example, a target can include a protein such as anaphylatoxins C3a and C5a which are described in: Humbles, et al. Nature 406, 998-1001 (2000); Kawamoto, et al., J Clin Invest, 114, 399 407 (2004); Gerard, et al. Complement in allergy and asthma. Curr Opin Immunol 14, 705-708 (2002); Ames, et al., J Biol Chem 271, 20231-20234 (1996); Fitch, et al., Circulation 100, 2499-2506 (1999); Shernan, et al., Ann Thorac Surg 77, 942-949 (2004).

As a further example, a target can include proteins, such as endothelial growth factors like VEGF, associated with diseases such as macular degeneration and cancers. As a further example, a target can include growth hormones such as human growth hormone, associated with acromegaly. In another example, targets such as creatine kinase, troponin I and troponin T are associated with myocardial infarction. In a further example, a target can be a protein of a pathogen such as a virus, bacterium, fungus, or single-celled organism. Thus, in some embodiments, the target can be the F1 and F2 subunits of respiratory syncytial virus fusion protein, or VP1 of Coxsackievirus A9 (CAV9), or Vpr of HIV. In some embodiments, the target can be a protein found in the exosporium of Bacillus anthracis, such as protein BclA. In other embodiments, the target can be one or more proteins making up a toxin such as botulinum neurotoxins of various serotypes (including heavy and light chains, as described for example in Singh, Nat. Struct. Biol., 2000, 7:617-9, and references therein), tetanus neurotoxin, or anthrax toxin (including lethal factor, protective antigen and edema factor, as reviewed for example in Stubbs, Trends Pharmcol Sci, 2002, 23:539-41, and references therein). In yet further embodiments, the target can be a polypeptide having a C-terminal sequence of EFYA. Additional examples of targets include other polypeptides used to treat or diagnose disease. Example polypeptides used to treat or diagnose disease include, for example, Enfuvirtide (commercially known as Fuzeon), interferons, monoclonal antibodies such as Rituximab (Rituxan), and the like.

The term “intermediate target” refers to a target that is different from the ultimately desired target but is sufficiently similar so as to aid in preparing the desired polypeptide of the invention. For example, the intermediate target can be a peptide fragment of the desired target, where the peptide fragment contains at least the last 3, 4, 5, or 6 amino acids at the carboxyl terminus (or N-terminus) of the desired target. Peptides can often be easier to manipulate than large proteins. In other embodiments, the intermediate target can be a target in which the C-terminus has about 20 to about 80 percent homology with the C-terminus (or N-terminus) of the ultimately desired target. In this way, in vitro evolution of the polypeptide containing a PDZ domain can be coaxed in a desired direction. Several different intermediate targets can be used in the in vitro evolution process. For example, intermediate targets having increasing percent identity can be used in successive rounds of evolution.

As used herein, the term “pathogen” refers to any microorganism, virus or prion causing disease in humans, other animals or plants, including commercially important domesticated animals and crops. Pathogens include, for example, bacteria such as Bacillus anthracis, Escherichia coli O:157, Yersinia pestis, Helicobacter pylori, Clostridium difficile, Streptococcus pneumoniae, Staphylococcus aureus, Mycobacterium tuberculosis, Mycobacterium bovis, Clostridium botulinum, Clostridium tetani and the like. Viral pathogens include, for example, human immunodeficiency viruses (HIV), hepatitis A, B and C viruses, (HAV, HBV, HCV), respiratory syncytial virus (RSV), poliovirus, Coxsackievirus A9 (CAV9), smallpox virus, CMV (cytomegalovirus), flaviviruses, papillomaviruses, coronaviruses (e.g., SARS-CoV), influenza virus, viral plant pathogens such as alfalfa mosaic virus, tobacco mosaic virus, and the like. Other microbial pathogens include parasites and fungi such as, for example, Plasmodium falciparum (malaria) and the fungus Candida albicans, respectively, and the like. Prion pathogens include transmissible spongiform encephalopathies such as bovine spongiform enceplialopathy (BSE), Creutzfeld-Jacob disease (CJD) and the like.

As used herein, an “enzyme” is defined as any of numerous proteins that catalyze specific chemical reactions. Examples of enzymes include β-lactamases, polymerases, proteases, endonucleases, glutathione S-transferase (GST), alkaline phosphatase, and the like. Many toxins, such as cholera toxin, botulin toxin and the like, are or comprise enzymes.

As used herein, a “fluorescent protein” is defined as a protein having ability to fluoresce in the visible wavelengths of the electromagnetic spectrum (i.e., from about 300 nm to about 700 nm). Examples of fluorescent proteins include the Green Fluorescent Protein (GFP) and its derivatives, as well as DsRed and other proteins and their derivatives available commercially from BD Biosciences under the trademark “Living Colors”.

As used herein, an “epitope” is defined as a molecular region of an antigen capable of eliciting an immune response and of combining with the specific antibody produced by such a response. Epitopes can be peptides, polynucleotides, polypeptides, polysaccharides and the like.

As used herein, the term “antibody” includes polyclonal antibodies and monoclonal antibodies as well as fragments thereof. Antibodies include, but are not limited to mouse, rat, and rabbit, human, chimeric antibodies and the like. The term “antibody” also includes antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. Proc. Natl. Acad. Sci., 1985, 82, 8653 or Spira, et al., J. Immunol. Methods, 1984, 74, 307.

The invention also provides fragments of the polyclonal and monoclonal antibodies described above. These “antibody fragments” typically retain some ability to selectively bind with its antigen or immunogen. Such antibody fragments can include, but are not limited to: Fab, Fab′, F(ab′)₂, Fv, and SCA. An example of a biologically active antibody fragment is a CDR region of the antibody. Methods of making these fragments are known in the art, see for example, Harlow and Lane (1988), infra.

The antibodies of this invention also can be modified to create chimeric antibodies and humanized antibodies (Oi, et al., BioTechniques, 1986, 4(3), 214 which is incorporated herein by reference in its entirety). Chimeric antibodies are, for example, those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies (Herlyn, et al., Science, 1986, 232:100, which is incorporated herein by reference in its entirety). An anti-idiotypic antibody is an antibody which recognizes unique determinants present on the monoclonal antibody produced by the hybridoma of interest.

Antibodies according to the present invention can also include genetically engineered antibody fragments. For example, molecular clones of variable domains of antibodies can be transformed into single-chain variable domains (scFv), diabodies, Fab (Barbas et al., Proc. Natl. Acad. Sci. USA, 1992, 9, 10164), bivalent Fab (Fab′), etc., using standard recombinant DNA technology. Phage display (Smith, Science, 1985, 228, 1315), ribosome display (Hanes & Pluckthun, Proc. Natl. Acad. Sci. USA, 1997, 94, 4937) and mRNA display (Xu et al., Chem. Biol, 2002, 9, 933) can be used in vitro to select antibodies with desired affinity and/or specificity.

Laboratory methods for producing polyclonal antibodies and monoclonal antibodies, as well as deducing their corresponding nucleic acid sequences, are known in the art, see, e.g., ANTIBODIES, A LABORATORY MANUAL (Harlow and Lane eds. (1988)) and Sambrook et al. MOLECULAR CLONING: A LABORATORY MANUAL, 2^{nd} edition (1989), each of which is incorporated herein by reference in its entirety. The monoclonal antibodies of the present invention can be biologically produced by introducing an antigen such as a protein or a fragment thereof into an animal, e.g., a mouse or a rabbit. The antibody producing cells in the animal are isolated and fused with myeloma cells or heteromyeloma cells to produce hybrid cells or hybridomas.

As used herein, “nucleic acids” or “polynucleotides” refer to polymeric forms of nucleotides or analogs thereof, of any length. The polynucleotides can contain deoxyribonucleotides, ribonucleotides, and/or their analogs. Nucleotides can have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, dsRNA, and the like.

Nucleic acid molecules further include oligonucleotides, such as antisense molecules, probes, primers and the like. Oligonucleotides typically have from about 2 to about 100, 8 to about 30, or 10 to about 28 nucleotides or analogs thereof.

Nucleic acid molecules can also contain modified backbones, modified bases, and modified sugars, such as for enhancing certain desirable properties such as in vivo stability, binding affinity, etc. Modifications of nucleic acids are well known in the art and include, for example, modifications described in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361, 5,625,050, 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437, 5,677,439, 5,539,082; 5,714,331, 5,719,262, 5,489,677, 5,602,240, 5,034,506, 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633, 5,700,920, 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617, 5,681,941, 5,750,692, 5,013,830; 5,149,797; 5,220,007; 5,256,775; 5,366,878; 5,403,711; 5,491,133; 5,565,350; 5,623,065; 5,652,355; 5,652,356; and 5,700,922, each of which is incorporated herein by reference in its entirety.

Isolation, preparation, and manipulation of nucleic acids, is well known in the art and is well described in Sambrook, et al., supra.

The present invention also relates to “vectors” which include the isolated DNA molecules of the present invention, “host cells” which are genetically engineered with the recombinant vectors, or which are otherwise engineered to produce the polypeptides of the invention, and the production of evolved PDZ domains, or derivatives thereof, by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In one embodiment, the DNA of the invention is operatively associated with an appropriate heterologous regulatory element (e.g., promoter or enhancer), such as, the phage lambda PL promoter, the E. coli lac, trp, and tac promoters, the SV40 early and late promoters and promoters of retroviral LTRs, to name a few. Other suitable promoters will be known to the skilled artisan.

In embodiments in which vectors contain expression constructs, these constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Selection of appropriate vectors and promoters for expression in a host cell is a well known procedure and the requisite techniques for expression vector construction, introduction of the vector into the host and expression in the host are routine skills in the art.

The present invention also relates to host cells containing the vector constructs discussed herein, and additionally encompasses host cells containing nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. The host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins.

Appropriate cell lines can be chosen to ensure the desired modifications and processing of the foreign protein expressed.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Sambrook et al., supra.

As used herein, the phrase “optimization” is intended to mean any process whereby a DNA sequence encoding a translation product (polypeptide or protein) is changed to improve the expression level of this protein without altering its amino acid sequence. For instance, gene optimization can be achieved by computational methods (e.g., Fuglsang, Protein Expr Purif. 2003, 31:247-9). An alternative method of gene optimization amounts to a specialized application of directed evolution described by Stemmer et al., Gene, 1993, 123:1-7. Example expression systems include bacteria (e.g., E. coli) and yeast.

As used herein, the term “cell-free selection” or “cell-free screening assay” is defined as any affinity selection method which does not involve the direct use of living cells. Examples of cell-free selections include ribosome display (Hanes & Pluckthun, Proc. Natl. Acad. Sci. USA, 1997, 94, 4937) and mRNA display (Xu et al., Chem. Biol, 2002, 9, 933). Phage display, which requires the transformation of DNA into cells in order to create selectable libraries, does not constitute an example of cell-free selection.

As used herein, the term “contacting” refers to the bringing together of designated substances (e.g., a sample and a polypeptide of the invention) such that the substances can interact at the molecular level sufficient to show, for example, binding affinity.

Methods of Preparing Polypeptides

The present invention further provides methods of preparing a polypeptide using in vitro evolution techniques. For example, a polypeptide containing a PDZ domain can be prepared by creating a library of polypeptides from one or more parent polypeptides also containing a PDZ domain. One or more polypeptides having improved binding affinity for a desired target can then be identified from the library. In some embodiments, the identified polypeptides can be used to create a further library from which another polypeptide can be identified, potentially having even greater binding affinity for the selected target. This process of mutagenesis and selection can be repeated iteratively several times.

Binding affinities (reported as dissociation constant, or K_{d}) of evolved PDZ domains in polypeptides of the invention can be from about 1 mM to about 1 fM, about 1000 nM to about 1 fM, about 100 nM to about 1 fM, 50 nM to about 1 fM, about 20 nM to about 1 fM, about 15 nM to about 1 fM, about 10 nM to about 1 fM, about 5 nM to about 1 fM or about 1 nM to about 1 fM. In some embodiments, the binding affinity of a PDZ domain according to the present invention for a preselected target is less than about 100 nM, less than about 50 nM, less than about 20 nM, less than about 15 nM or less than about 10 nM. Affinity can be measured by surface plasmon resonance (SPR) as implemented, for example, on a Biacore instrument (Biacore). Identification of library members that bind to the desired target can be carried out by any suitable methods. In some embodiments, polypeptides can be identified by phage display, or in a cell-free selection such as mRNA display.

In further embodiments, the present invention provides a method of preparing a polypeptide containing a PDZ domain by forming a library of polypeptides from one or more parent polypeptides comprising a PDZ domain; selecting a first selected polypeptide from the library, where the first selected polypeptide has binding affinity to an intermediate target. The intermediate target can have, for example, 20% to 80% (e.g., 20%, 30%, 40%, 50%, 60%, 70% or 80%) sequence identity in the last 5 amino acids (e.g., C-terminal) with the last 5 amino acids (e.g., C-terminal) of the desired target. A further library of polypeptides can then be created from the first selected polypeptide and the process can be repeated until a library yields a polypeptide capable of binding with the desired target. The intermediate target can act as an evolutionary guide for the evolving polypeptide, and can be particularly useful when the C-terminal sequence of the target is substantially different from the C-terminal sequence of the natural binder to the parent polypeptide. One or more intermediate targets can be used, and different intermediate targets can be used for each iteration.

Therapeutic and Prophylactic Methods

Methods of treatment according to the present invention can include both prophylaxis and therapy. Prophylaxis or therapy can be accomplished by administration to a patient of therapeutic agents such as polypeptides containing PDZ domains prepared, for example, by the directed evolution methods described herein. In some embodiments, methods of treatment include administration of a polypeptide of the invention. In other embodiments, methods of treatment include administration of a peptide which can be bound by a polypeptide of the invention. The therapeutic agent can be administered at a single time point or multiple time points to a single or multiple sites. Administration can also be nearly simultaneous to multiple sites. Patients or subjects include mammals, such as human, bovine, equine, canine, feline, porcine, and ovine animals. The subject is preferably a human.

A disease or disorder, such as a viral infection, cancer, allergy, or other pathological condition associated with a target, can be diagnosed using criteria generally accepted in the art, including, for example, the presence of a malignant tumor or elevated white blood cell count. Therapeutic agents can be administered either prior to or following surgical removal of primary tumors and/or treatment such as administration of radiotherapy or conventional chemotherapeutic drugs. In further embodiments, therapeutic agents such as polypeptide of the invention can also be administered prior to infection by an infectious agent such as a virus, bacteria, or other pathogen.

Within certain embodiments, therapy can be immunotherapy, which can be active immunotherapy in which treatment relies on the in vivo stimulation of the endogenous host immune system (e.g., stimulation of endogenous effector cells) to react against tumors or infected cells with the administration of binding proteins prepared according to the methods described herein. Within other embodiments, immunotherapy can be passive immunotherapy, in which treatment involves the delivery of agents with, for example, immune reactivity (such as evolved PDZ domains fused to an Fc domain or conjugated to an antibody or antibody fragment) that can directly or indirectly mediate antitumor, anti-inflammatory, or other effects and do not necessarily depend on an intact host immune system. Examples of effector cells include T cells, T lymphocytes (such as CD8+ cytotoxic T lymphocytes and CD4+ T-helper tumor-infiltrating lymphocytes), killer cells (such as Natural Killer cells and lymphokine-activated killer cells), B cells and antigen-presenting cells (such as dendritic cells and macrophages).

The therapeutic agents prepared according to the methods described herein can be combined with a pharmaceutically acceptable carrier to produce a pharmaceutical composition. As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers ate formulated by well known conventional methods (see, for example, Remington's Pharmaceutical Sciences, Chapter 43, 14th Ed., Mack Publishing Co, Easton Pa. 18042, USA).

Therapeutic and Prophylactic Compositions and Uses

Much like antibodies and antibody fragments, polypeptides containing PDZ domains and their derivatives can be useful in the treatment of numerous disorders including, for example, cancer, inflammatory disorders, such as adult respiratory distress syndrome (ARDS), hypovolemic shock, ulcerative colitis, rheumatoid arthritis, and others, as shown in Table 1 which provides a list of diseases and molecular targets addressed by therapeutic antibodies.

TABLE 1
Monoclonal antibody-based therapeutics
(Nature Biotechnology, 2003, 21, 868).

| | | Year |
|---|---|---|
| Product | Initial indication | approved |
| Bexxar (tositumomab; radiolabelled monoclonal | Treatment of CD20 positive | 2003 (US) |
| antibody directed against CD20, produced in a | follicular non-Hodgkin | |
| mammalian cell line.) | lymphoma | |
| Xolair (Omalizumab; rIgG1k Mab that binds | Asthma | 2003 (US) |
| IgE, produced in CHO cells) | | |
| Humira (adalimumab; r human Mab (antiTNF) | Rheumatoid arthritis | 2002 (US) |
| created using phage display technology and | | |
| produced in a mammalian cell line) | | |
| Zevalin (Ibritumomab Tiuxetan; murine Mab | Non-Hodgkin lymphoma | 2002 (US) |
| produced in a CHO cell line, targeted against the | | |
| CD20 antigen. A radiotherapy agent.) | | |
| Mabcampath (EU) or Campath (US) | Chronic lymphocytic leukemia | 2001 (EU, |
| (alemtuzumab; a humanized monoclonal | | US) |
| antibody directed against CD52 surface antigen | | |
| of B-lymphocytes.) | | |
| Mylotarg (gemtuzumab zogamicin; a humanized | Acute myeloid leukemia | 2000 (US) |
| antibody-toxic antibiotic conjuage targeted | | |
| against CD33 antigen found on leukemic blast | | |
| cells.) | | |
| Herceptin (trastuzumab, humanized antibody | Treatment of metastatic breast | 1998 (US), |
| directed against human epidermal growth factor | cancer if tumor overexpresses | 2000 (EU) |
| receptor 2 (HER2)) | HER2 protein | |
| Remicade (infliximab, chimeric mAb directed | Treatment of Crohn disease | 1998 (US), |
| against TNF-alpha | | 1999 (EU) |
| Synagis (palivizumab, humanized mAb directed | Prophylaxis of lower | 1998 (US), |
| against an epitope on the surface of respiratory | respiratory disease caused by | 1999 (EU) |
| syncytial virus.) | syncytial virus in pediatric | |
| | patients | |
| Zenapax (daclizumab, humanized mAb directed | Prevention of acute kidney | 1997 (US), |
| against the alpha-chain of the IL-2 receptor) | transplant rejection | 1999 (EU) |
| Humaspect (Votumumab, human mAb directed | Detection of carcinoma of the | 1998 (EU) |
| against cytokeratin tumor-associated antigen) | colon or rectum | |
| Mabthera (Rituximab, chimeric mAb directed | Non-Hodgkin lymphoma | 1998 (EU) |
| against CD20 surface antigen of B lymphocytes. | | |
| See also Rituxan.) | | |
| Simulect (basiliximab, chimeric mAb directed | Prophylaxis of acute organ | 1998 (EU) |
| against the alpha-chain of the IL-2 receptor) | rejection in allogeneic renal | |
| | transplantation | |
| LeukoScan (Sulesomab, murine mAb fragment | Diagnostic imaging for | 1997 (EU) |
| (Fab) directed against NCA 90, a surface | infection/inflammation in bone | |
| granulocyte nonspecific cross-reacting antigen.) | of patients with osteomyelitis | |
| Rituxan (rituximab chimeric mAb directed | Non-Hodgkin lymphoma | 1997 (US) |
| against CD20 antigen found on the surface of B | | |
| lymphocytes) | | |
| Verluma (Nofetumomab murine mAb fragments | Detection of small-cell lung | 1996 (US) |
| (Fab) directed against carcinoma-associated | cancer | |
| antigen.) | | |
| Tecnemab KI (murine mAb fragments (Fab/Fab2 | Diagnosis of cutaneous | 1996 (EU) |
| mix) directed against HMW-MAA) | melanoma lesions | |
| ProstaScint (capromab-pentetate, murine mAb | Detection/staging/ follow-up of | 1996 (US) |
| directed against the tumor surface antigen | prostate adenocarcinoma | |
| PSMA) | | |
| MyoScint (imiciromab-pentetate, murine mAb | Myocardial infarction imaging | 1996 (US) |
| fragment directed against human cardiac myosin) | agent | |
| CEA-scan (arcitumomab, murine mAb fragment | Detection of | 1996 (US, |
| (Fab), directed against human carcinoembryonic | recurrent/metastatic colorectal | EU) |
| antigen, CEA) | cancer | |
| Indimacis 125 (Igovomab, murine mAb fragment | Diagnosis of ovarian | 1996 (EU) |
| (Fab2) directed against the tumor-associated | adenocarcinoma | |
| antigen CA 125) | | |
| ReoPro (abciximab, Fab fragments derived from | Prevention of blood clots | 1994 (US) |
| a chimeric mAb, directed against the platelet | | |
| surface receptor GPIIb/IIIa) | | |
| OncoScint CR/OV (satumomab pendetide, | Detection/staging/follow-up of | 1992 (US) |
| murine mAb directed against TAG-72, a tumor- | colorectal and ovarian cancers | |
| associated glycoprotein) | | |
| Orthoclone OKT3 (Muromomab CD3, murine | Reversal of acute kidney | 1986 (US) |
| mAb directed against the T-lymphocyte surface | transplant rejection | |
| antigen CD3) | | |

Therapeutic formulations of polypeptides of the invention or derivatives thereof can be prepared for storage by mixing the polypeptide or derivative thereof having the desired degree of purity with optional pharmaceutically acceptable carriers, excipients, or stabilizers (see, e.g., Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumins gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Polypeptides of the invention or derivatives thereof for in vivo administration are preferably sterile. This can be readily accomplished by filtration through sterile filtration membranes, prior to or following lyophilization and reconstitution. The polypeptides of the invention or derivatives thereof ordinarily will be stored in lyophilized form or in solution.

Therapeutic polypeptide compositions generally are placed into a container having a sterile access port, for example, an intravenous solution bag or vial having a stopper pierceable by a hypodermic injection needle.

The route of polypeptide administration can be carried out in accord with known methods, e.g., inhalation, injection or infusion by intravenous, intraperitoneal, intracerebral, intramuscular, intraocular, intraarterial, or intralesional routes, by enema or suppository, or by sustained release systems as noted below. The polypeptide or its derivative is given systemically or at a site of inflammation.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919 and EP 58,481), copolymers of L-glutamic acid and gamma ethyl-L-glutamate (Sidman et al., Biopolymers, 1983, 22, 547), poly (2-hydroxyethyl-methacrylate) (Langer et al., J. Biomed. Mater. Res., 1981, 15, 167 and Langer, Chem. Tech., 1982, 12, 98), ethylene vinyl acetate (Langer et al., supra) or poly-D-(−)-3-hydroxybutyric acid (EP 133,988). Sustained-release compositions also include liposomally entrapped evolved PDZ domain or derivative thereof. Liposomes containing an evolved PDZ domain or derivative thereof can be prepared by methods known per se: DE 3,218,121; Epstein et al., Proc. Natl. Acad. Sci. U.S.A., 1985,82, 3688; Hwang et al., Proc. Natl. Acad. Sci. U.S.A., 1980, 77, 4030; EP 52,322; EP 36,676; EP 88,046; EP 143,949; EP 142,641; U.S. Pat. Nos. 4,485,045 and 4,544,545; and EP 102,324. Ordinarily the liposomes are of the small (about 200-800 Angstroms) unilamelar type in which the lipid content is greater than about 30 mole percent cholesterol, the selected proportion being adjusted for the most efficacious therapy.

An “effective amount” of a polypeptide of the invention to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect. Typically, the clinician will administer the polypeptide until a dosage is reached that achieves the desired effect. The progress of this therapy is easily monitored by conventional assays.

In the treatment and prevention of a disease or disorder, the polypeptide composition can be formulated, dosed, and administered in a fashion consistent with good medical practice. Factors for consideration in this context include the particular disorder being treated, the particular mammal being treated, the clinical condition of the individual patient, the cause of the disorder, the site of delivery of the polypeptide, the particular type of polypeptide, the method of administration, the scheduling of administration, and other factors known to medical practitioners. The “therapeutically effective amount” of polypeptide to be administered can be governed by such considerations, and is the minimum amount necessary to prevent, ameliorate, or treat the inflammatory disorder. Such amount is preferably below the amount that is toxic to the host or renders the host significantly more susceptible to infections.

As a general proposition, the initial pharmaceutically effective amount of the polypeptide administered parenterally per dose can be in the range of about 0.1 to 50 mg/kg of patient body weight per day, with the typical initial range of polypeptide used being 0.3 to 20 mg/kg/day, more preferably 0.3 to 15 mg/kg/day. As noted above, however, these suggested amounts of polypeptide are subject to therapeutic discretion.

The polypeptide of the invention need not be, but is optionally formulated with one or more agents currently used to prevent or treat the disease or disorder in question. For example, in rheumatoid arthritis, a polypeptide can be given in conjunction with a glucocorticosteroid, or for cancer, a polypeptide can be given in conjunction with a chemotherapeutic. The polypeptide can also be formulated with one or more other polypeptides of the invention to provide a therapeutic “cocktail.”

Methods of Detection

The invention further provides a method for detecting a disease, disease-causing pathogen or disorder such as cancer in a sample, comprising contacting the sample with a polypeptide containing a PDZ domain that binds to a target in the sample, where the target is associated with the disease, pathogen, or disorder. The target can be, for example, a nucleic acid or protein encoded thereby. The target can be a substance, such as a peptide or protein that is produced directly or indirectly by a pathogen, including their toxins and the like. The sample can be an environmental sample, or a tissue from a mammal, such as human, bovine, equine, canine, feline, porcine, and ovine tissue. In some embodiments, the tissue is human. The tissue can comprise a tumor specimen, cerebrospinal fluid, or other suitable specimen such a tissue likely to contain the target of interest. In one embodiment, the method comprises use of an ELISA type assay that employs an evolved PDZ domain or derivative thereof by the methods described herein to detect the presence of target in a specimen. This method can also be used to monitor target levels in a tissue sample of a patient. For example, the suitability of a therapeutic regimen for initial or continued treatment can be determined by monitoring target levels according to this method.

The invention further provides a method for detecting a disease, including a disease-causing pathogen or a disease such as cancer, in a patient by administering a polypeptide of the invention to the patient and detecting binding of the polypeptide in the patient. In some embodiments, the administered polypeptide further contains a reporter group, such as a radioactive moiety, chelated heavy metal, or other imaging agent to facilitate detection of binding of the polypeptide in the patient. Binding of polypeptide in the patient can be observed as localization of the polypeptide in certain tissues containing the desired target. For example, a polypeptide of the invention that is capable of specifically binding to a cancer marker such as a polypeptide differentially expressed from certain cancer cells can reveal the presence of a tumor or diseased tissue by detection of localization of the polypeptide. Methods for scanning a patient, such as a human patient, are well known in the art and include radiography, MRI, and related techniques.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g., Sambrook, et al., Molecular Cloning: A Laboratory Manual, 2nd edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson et al., eds. (1995)); Antibodies, A Laboratory Manual (Harlow and Lane eds. (1988)); Animal Cell Culture (R. I. Freshney ed. (1987)); and Phage Display: A Laboratory Manual (C. F. Barbas III et al., (2001)), each of which is incorporated herein by reference in its entirety.

Methods of Purification

The present invention further provides a method of purifying a protein comprising contacting said protein with an immobilized polypeptide containing a PDZ domain, wherein the immobilized polypeptide has binding affinity for the protein. Suitable binding affinities (reported as dissociation constant, or K_{d}) include from about 1 mM to about 1 fM, about 1000 nM to about 1 fM, about 100 nM to about 1 fM, 50 nM to about 1 fM, about 20 nM to about 1 fM, about 15 nM to about 1 fM, about 10 nM to about 1 fM, about 5 nM to about 1 fM or about 1 nM to about 1 fM. In some embodiments, the binding affinity is less than about 100 nM, less than about 50 nM, less than about 20 nM, less than about 15 nM or less than about 10 nM.

The following examples are presented to illustrate the present invention and to assist one of ordinary skill in making and using the same. The examples are not intended in any way to otherwise limit the scope of the invention.

### EXAMPLES

### Example 1

### Synthesis of Human Cask PDZ Domain Gene Optimized for Expression in Escherichia coli and Saccharomyces cerevisiae

A gene fragment, hCASK-PDZopt, having the sequence shown in SEQ ID NO: 1 is obtained from a commercial supplier (GENEART, Germany). This hCASK-PDZopt codes for the PDZ domain of the human hCASK gene (GenBank accession number AF032119) product. The sequence of this PDZ domain is provided in SEQ ID NO: 2. The gene fragment of SEQ ID NO:1 is designed for optimal expression in both Escherichia coli and Saccharomyces cerevisiae. The gene fragment is cloned into vector pCR-Script Amp (Stratagene, LaJolla, Calif.) using KpnI and SacI restriction sites and transformed into E. coli XL10-Gold (Stratagene). DNA sequencing using standard labeled-dideoxy terminator chemistry and an Applied BioSystems instrument is carried out to verify the sequence of the cloned synthetic gene.

### Example 2

### Construction and Expression of Translational Fusion of GST and Human hCASK PDZ Synthetic Gene

The synthetic gene of SEQ ID NO:1, hCASK-PDZopt, is sub-cloned from the pCR-Script vector of Example 1 into plasmid pGEX-2TK (Amersham Biosciences) using EcoRI and BamHI restriction sites. This yields plasmid pGEX-hCASK-PDZopt, comprising a translational fusion whose open reading frame includes the GST gene fused to the synthetic PDZ domain gene fragment. DNA sequencing is carried out according to standard methods to confirm that the DNA sequence of the subclone codes for the protein provided in SEQ ID NO: 3, namely GST fused to the hCASK PDZ domain.

pGEX-hCASK-PDZopt DNA is transformed into E. coli strain JM109 for expression of the GST-hCASK PDZ fusion protein and purification via affinity chromatography using glutathione sepharose affinity medium (Amersham Biosciences). Purified protein is visualized by coomassie blue-stained SDS-PAGE.

Function of the GST moiety is confirmed by incubating 1 μg of the fusion protein with 1-chloro-2,4-dinitrobenzene (CDNB, provided by Amersham Biosciences) and reduced glutathione, as described by the manufacturer, in 0.1M potassium phosphate buffer, pH 6.5, and monitoring absorbance at 340 nm. Increase of absorbance of >0.02 OD/min in the first 5 minutes of the reaction is expected for functional GST-CASK fusion protein.

### Example 3

### Construction and Expression of Translational Fusion of Alkaline Phosphatase Gene and Human hCASK PDZ Synthetic Gene

The synthetic gene of SEQ ID NO: 1, hCASK-PDZopt, is fused with the alkaline phosphatase gene of Escherichia coli (phoA) via overlap PCR, a well-known technique (Horton et al., 1990, Biotechniques, 8, 528), to yield a gene encoding the polypeptide shown in SEQ ID NO: 4. The 5′ primer used to amplify the hCASK-PDZopt gene fragment encodes a sequence of amino acids corresponding to the signal sequence shown in SEQ ID NO: 4. The outer-most primers are designed to provide convenient restriction sites (NcoI and HindIII) for cloning the gene coding into plasmid pQE-60 (Qiagen, www.qiagen.com) digested with NcoI and HindIII restriction sites. This yields plasmid pQE-hCASK-PDZopt-alkphos, comprising a translational fusion whose open reading frame includes the alkaline phosphatase gene of Escherichia coli fused to the synthetic PDZ domain gene fragment. DNA sequencing is carried out according to standard methods to confirm that the DNA sequence of the subclone codes for the protein provided in SEQ ID NO: 4.

pQE-hCASK-PDZopt-alkphos DNA is transformed into E. coli strain JM109 for expression of the alkaline phosphatase-hCASK PDZ fusion protein and purification via affinity chromatography using streptavidin-sepharose (Amersham Biosciences) to which the N-terminal-biotinylated peptide ligand of hCASK PDZ (obtained from any of many custom peptide suppliers such as Invitrogen) is bound. Purified protein is visualized by coomassie blue-stained SDS-PAGE. Function of the alkaline phosphatase moiety is confirmed by incubating 1 μg of the fusion protein with para-nitrophenol phosphate colorimetric substrate (cat. No. A3469, Sigma, St-Louis, Mo.) monitoring absorbance at 405 nm. Increase of absorbance above background is expected. In contrast, a control such as GST-CASK fusion protein incubated in the same conditions is expected to yield a change of absorbance similar to background (i.e., substrate alone).

### Example 4

### Construction and Expression of Translational Fusion of Immunoglobulin Fc Gene and Synthetic Human hCASK PDZ Synthetic Gene

The synthetic gene of SEQ ID NO: 1, hCASK-PDZopt, is fused with a human immunoglobulin Fc gene fragment by overlap PCR, a well-known technique (Horton et al., 1990, Biotechniques, 8, 528), to yield a gene encoding the polypeptide shown in SEQ ID NO: 5. The 5′ primer used to amplify the hCASK-PDZopt gene fragment encodes a sequence of amino acids corresponding to the signal sequence of human light chain immunoglobulin shown in SEQ ID NO: 5. The outer-most primers are designed to provide convenient restriction sites for cloning the gene coding into plasmid pCDNA3.1(+)myc/his/LacZ (Qiagen, www.qiagen.com) digested with HindIII and PmeI restriction sites. This yields plasmid pCDNA-hCASK-PDZopt-Fc, comprising a translational fusion whose open reading frame includes the human immunoglobulin Fc gene fragment fused to the synthetic PDZ domain gene fragment. DNA sequencing is carried out according to standard methods to confirm that the DNA sequence of the subclone codes for the protein provided in SEQ ID NO: 5.

Plasmid pCDNA-hCASK-PDZopt-Fc, linearized away from the hCASK PDZ-Fc fusion gene using a unique restriction site, is transfected according to well-known procedures (Sambrook & Russell, 2001, Molecular cloning: a laboratory manual) into mammalian cell line NS0 approvable for the production of recombinant immunoglobulins for therapeutic use. Stable transfectants are screened for clones producing useful amounts of the fusion protein. Fusion protein produced in this fashion can be isolated from the culture medium and purified using standard antibody affinity purification resins such as Protein G sepharose (Amersham Biosciences). The protein can be assayed for biological activity or ability to bind a ligand.

### Example 5.1

### Use of GST-hCASK PDZ Variant Fusion as an Affinity Reagent (Western Blotting and ELISA.)

Variants of purified GST-hCASK PDZ fusion protein such as those described in examples 10 and 13, (see also example 14) are used as an affinity reagent to detect proteins which bind to the PDZ moiety of the fusion protein. In this example, the affinity matured GST-hCASK PDZ fusion of example 12 is used to detect syndecan-2. The GST moiety acts as an epitope tag, or reporter domain. To detect syndecan-2 in human brain tissues, the brain tissues are homogenized, suspended in SDS-PAGE reducing sample buffer (Fermentas) and boiled for 3 minutes. The samples are resolved by SDS-PAGE and western transfer is carried out to blot the separated proteins onto a membrane according to standard methods. The blot is then blocked with I-block (Applied Biosystems) according to instructions from the manufacturer and probed using affinity-matured GST-hCASK PDZ fusion protein. The membrane is washed and probed with a secondary antibody specific to GST and labeled with horseradish peroxidase (Amersham Biosystems). Chemiluminescence is used to detect the secondary antibody according to chemilumenescence kit manufacturer protocols (Vector Labs).

To detect the protein without electrophoretic separation, an ELISA is carried out. In this example, affinity-matured GST-hCASK PDZ is immobilized on the bottom of the wells of an ELISA plate (1 μg/well). The wells of the plate are then blocked with I-block (Applied Biosystems), washed with buffer, and a brain homogenate sample is added to the well. The samples are allowed to incubate for 2 hours at room temperature. The plate is washed, and the presence of syndecan-2 is determined by using a secondary antibody specific to syndecan-2 (Zymed laboratories, www.zymed.com), and, following incubation and wash, a tertiary goat anti-rabbit antibody labeled with horseradish peroxidase (VWR, www.vwr.com). Chemiluminescence is used to detect the tertiary antibody—and indirectly, the target syndecan-2—according to chemiluminescence kit manufacturer protocols (Vector Labs).

### Example 5.2

### Use of Alkaline Phosphatase-hCASK PDZ Fusion as an Affinity Reagent (Western Blotting and ELISA.)

Purified alkaline phosphatase-CASK PDZ fusion protein of example 3, or variants of this protein such as described in examples 10 and 13, (see also example 14) is used as an affinity reagent to detect proteins that bind to the PDZ moiety of the fusion protein. As explained in example 5.1, the protein to be detected is bound to a solid support, either via western transfer, or via direct or indirect adsorption to one or more wells of a multi-well assay plate (ELISA plate). Instead of using an anti-GST antibody for detection, as was done in example 5.1, binding of the PDZ domain or variants thereof is detected via the alkaline phosphatase reporter domain fused to the PDZ domain. Detection is carried out using Vector labs' DuoLuX chemiluminescent/fluorescent substrate for alkaline phosphatase according to the manufacturer's recommendations.

### Example 6

### Error-Prone PCR Mutagenesis of hCASK PDZ Gene

The synthetic gene fragment of SEQ ID NO: 1, hCASK-PDZopt, was excised from vector pCR-Script with SfiI and NotI restriction enzymes and ligated into the pre-digested pCANTAB5E phagemid (Amersham Biosciences) using Fast-Link DNA ligation kit (Epicentre Technologies, Madison, Wis.). The ligated DNA was transformed into electroporation-competent E. Coli XL1-Blue. Phage displaying the PDZ domain were rescued using helper phage according to standard methods, except that helper phage-infected cells were grown overnight at 30° C. instead of the usual 37° C. A phage ELISA was performed, confirming that recombinant phage displaying CASK PDZ domain bind specifically to its cognate peptide ligand (QKAPTKEFYA (SEQ ID NO: 13). DNA sequencing of the pCANTAB-hCASK-PDZopt construct was also carried out, ensuring that the sequence of the construct was as expected.

The hCASK-PDZopt gene was mutated by error-prone PCR (Leung et al., 1989, Technique, 1: 11-15), yielding a mutant library containing over 10⁶ unique mutants. Primers pCAN5′ (CATGATTACGCCAAGCTTTGG; SEQ ID NO: 14) and pCAN3′ (CGATCTAAAGTTTTGTCGTC; SEQ ID NO: 15) were used to amplify the PDZ gene under mutagenic conditions. To prepare the library, the mutated PCR product was digested with SfiI and NotI and ligated into the pCANTAB5E phagemid vector (Amersham Biosciences) using Fast-Link DNA ligation kit (Epicentre Technologies). The ligated DNA was then transformed into electroporation-competent E. coli XL1-Blue. Several frozen stocks were made from cultures of the mutant libraries and stored at −80° C. for future use. The complexity of the library was determined by counting the number of colonies obtained after plating an aliquot of the freshly transformed cells on agar-containing SOB medium, glucose, tetracycline, and ampicillin. A complexity of over 3×10⁶ unique clones was determined. The expected mutation rate should be about 1 to 3 amino acid substitutions per gene as determined by DNA sequencing of randomly picked clones.

### Example 7

### Random Combinatorial Mutagenesis of hCASK PDZ Gene

Amino acids likely to affect the specificity of the hCASK-PDZ gene product were identified by inspection of the crystal structure of hCASK PDZ, PDB number 1KWA (Daniels et al., Nat Struct Biol. 1998, 5, 317-25), using freely available Viewerlite 4.2 software (www.accelrys.com). Residues M501, I503, L505, Q553, L556 and R557 were identified as being in close contact with the C-terminal residue of the peptide recognized by hCASK PDZ (numbering scheme is according to Daniels et al., 1998, 5, 317-325). These residues were selected for randomization via combinatorial mutagenesis to create a single library in which any of the 20 amino acids can be found at these mutated positions in individual variants.

Codons corresponding to amino acids M501, I503, L505, Q553, L556 and R557 of the hCASK PDZ gene are mutated by amplification of the gene using primers pCAN5′ (CATGATTACGCCAAGCTTTGG) and NNK1B (CAATGATTCAATTCATTCATTTTMNNGGTMNNGGTMNNACCMNNTGGTTCATCGGTATTTTTTTG; SEQ ID NO: 16), NNK2A (AAAATGAATGAATTGAATCATTG; SEQ ID NO: 17) and NNK2B:(GGTAATAGAACCACGCATTTCMNNMNNCATTTTMNNCAATTGTTCAACGGTTTGA TTGG; SEQ ID NO: 18), as well as NNK3A (GAAATGCGTGGTTCTATTACC; SEQ ID NO: 19), and pCAN3′ (CGATCTAAAGTTTTGTCGTC) to generate three overlapping PCR products: codons M501, 1503, L505 are randomized in the first product, and codons encoding residues Q553, L556 and R557 are randomized in the second. Overlap PCR is carried out (Horton et al., 1990, Biotechniques, 8, 528) using the three purified PCR products to produce a pool of mutant genes of the following degenerate sequence:

(SEQ ID NO:20)

“CATGATTACGCCAAGCTTTGGAGCCTTTTTTTTGGAGATTTTCAACGTG
AAAAAATTATTATTCGCAATTCCTTTAGTTGTTCCTTTCTATGCGGCCCA
GCCGGCCGGATCCGGTATGGATATGGAAAATGTTACCCGTGTTCGTTTAG
TTCAATTTCAAAAAAATACCGATGAACCANNKGGTNNKACCNNKAAAATG
AATGAATTGAATCATTGTATTGTTGCCCGTATTATGCATGGTGGTATGAT
TCATCGTCAAGGTACTTTGCATGTTGGTGATGAAATTCGTGAAATTAATG
GTATTTCTGTTGCCAATCAAACCGTTGAACAATTGNNKAAAATGNNKNNK
GAAATGCGTGGTTCTATTACCTTTAAAATTGTTCCATCTTATCGTACCCA
ATCTTCTTCTGGAATTCATGCGGCCGCAGGTGCGCCGGTGCCGTATCCGG
ATCCGCTGGAACCGCGTGCCGCATAGACTGTTGAAAGTTG”,

where N signifies any of the four nucleotides, A, C, G or T, and K signifies either of the two nucleotides G or T. The underlined sequence codes for the PDZ domain and the bold codons (NNK) are degenerate. To prepare a library of combinatorial mutants, the mutated PCR product is digested with SfiI and NotI and ligated into the pCANTAB5E phagemid vector (Amersham Biosciences) using Fast-Link DNA ligation kit (Epicentre Technologies). The ligated DNA is then transformed into electroporation-competent E. coli XL1-Blue. Several frozen stocks are made from cultures of the mutant libraries and stored at −80° C. for future use. The complexity of the library (over 10⁷ unique clones) is determined by counting the number of colonies obtained after plating an aliquot of the freshly transformed cells on agar-containing LB medium and ampicillin. Presence of the expected mutations is verified by DNA sequencing of randomly picked clones.

### Example 9

### Target Set Mutagenesis of hCASK PDZ Gene

Amino acids likely to determine the specificity of the hCASK-PDZ gene product were identified by inspection of the crystal structure of hCASK PDZ, PDB number 1KWA (Daniels et al., Nat Struct Biol. 1998, 5, 317-25), using freely available Viewerlite 4.2 software (www.accelrys.com). Residues M501, I503, L505, Q553, L556 and R557 were identified as being in close contact with the C-terminal residue of the peptide recognized by hCASK PDZ. These residues were selected for Target Set Mutagenesis (Goldman and Youvan, Biotechnology (N Y), 1992, 10, 1557-61) to create a single library in which a subset, or target set, of the 20 amino acids is encoded at each mutated codon.

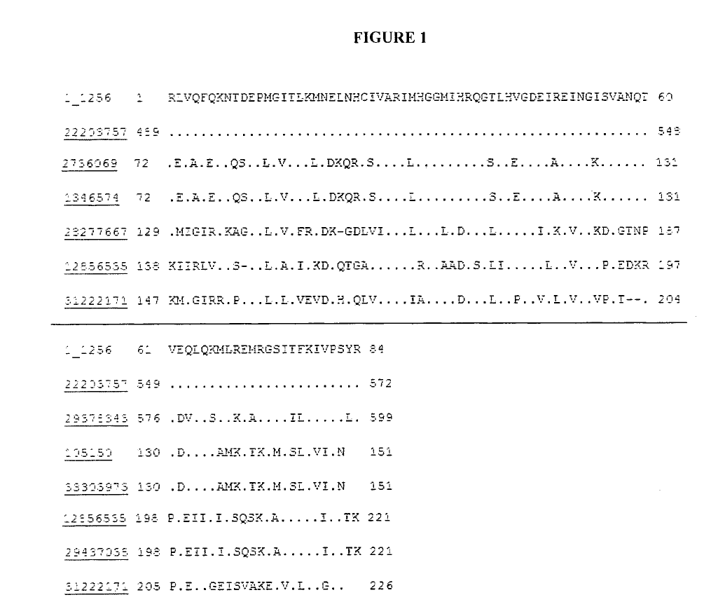

Each target set corresponds to the amino acids encountered at homologous positions in an alignment of related PDZ domains. The sequence alignment, shown in FIG. 1, was obtained by using the amino acid sequence of the hCASK PDZ domain as a query in a BLAST search of the non-redundant protein sequence database (http://www.ncbi.nlm.nih.gov/). Six different target sets were determined based on this alignment: for residue 501, amino acids M or L; for residue 503, amino acids 1. L, V, or A; for residue 505, amino acids V, I, L, or F; for residue 553, amino acids I or Q; for residue 556, amino acids L, I or M; for residue 557, amino acids R, K, or S. For each of these target sets, a degenerate codon is computed using the program Cyberdope (Kairos Scientific, San Diego, Calif.): for residue 501, the degenerate codon MTG yields amino acids M or L; for residue 503, the degenerate codon VYT yields amino acids I, L, T, P, V, or A; for residue 505, the degenerate codon NTT yields amino acids I, L, V, or F; for residue 553, the degenerate codon MWK yields amino acids I, K, L, M, N, H or Q; for residue 556, the degenerate codon MTK yields amino acids I, L, or M; for residue 557, the degenerate codon ARK yields amino acids R, K, S or N. (The encoded amino acids do not always match exactly the target set due to the structure of the genetic code.) Where A=adenosine, C=cytidine, G=guanosine, T=thymidine, B=C or G or T, D=A or G or T, H=A or C or T, K=G or T, M=A or C, N=A or C or G or T, R=A or G, S═C or G, V=A or C or G, W=A or T, Y=C or T, according to the IUPAC code. Oligonucleotides are then synthesized encoding the degenerate codons.

Codons corresponding to amino acids M501, I503, L505, Q553, L556 and R557 of the hCASK PDZ gene are mutated by amplification of the gene using oligonucleotide primers pCAN5′ (CATGATTACGCCAAGCTTTGG) and TSM1B (CAATGATTCAATTCATTCATTTTAANGGTARBACCCAKTGGTTCATCGGTATTTTTTTG; SEQ ID NO: 21), TSM2A (AAAATGAATGAATTGAATCATTG; SEQ ID NO: 22) and TSM2B (GGTAATAGAACCACGCATTTCMYTMAKCATTTTMWKCAATTGTTCAACGGTTTGATTGG; SEQ ID NO: 23), as well as TSM3A (GAAATGCGTGGTTCTATTACC; SEQ ID NO: 24), and pCAN3′ (CGATCTAAAGTTTTGTCGTC) to generate three overlapping PCR products: codons M501, 1503, L505 are mutated in the first product, and codons encoding residues Q553, L556 and R557 are mutated in the second. Overlap PCR is carried out (Horton et al., 1990, Biotechniques, 8, 528) using the three purified PCR products to produce a pool of mutant genes of the following degenerate sequence: “CATGATTACGCCAAGCTTTGGAGCCTTTTTTTTGGAGATTTTCAACGTGAAAAAATTATTAT TCGCAATTCCTTTAGTTGTTCCTTTCTATGCGGCCCAGCCGGCCGGATCCGGTATGGATATGG AAAATGTTACCCGTGTTCGTTTAGTTCAATTTCAAAAAAATACCGATGAACCAMTGGGTVY TACCNTTAAAATGAATGAATTGAATCATTGTATTGTTGCCCGTATTATGCATGGTGGTATGA TTCATCGTCAAGGTACTTTGCATGTTGGTGATGAAATTCGTGAAATTAATGGTATTTCTGTTG CCAATCAAACCGTTGAACAATTGMWKAAAATGMTKARKGAAATGCGTGGTTCTATTACCT TTAAAATTGTTCCATCTTATCGTACCCAATCTTCTTCTGGAATTCATGCGGCCGCAGGTGCGC CGGTGCCGTATCCGGATCCGCTGGAACCGCGTGCCGCATAGACTGTTGAAAGTTG”, (SEQ ID NO: 25) where A=adenosine, C=cytidine, G=guanosine, T=thymidine, B=C or G or r, D=A or G or T, H=A or C or T, K=G or T, M=A or C, N=A or C or G or T, R=A or G, S=C or G, V=A or C or G, W=A or T, Y=C or T, according to the IUPAC code. The underlined sequence codes for the PDZ domain and the bold codons are degenerate. To prepare a library of combinatorial mutants, the mutated PCR product is digested with SfiI and NotI and ligated into the pCANTAB5E phagemid vector (Amersham Biosciences) using Fast-Link DNA ligation kit (Epicentre Technologies). The ligated DNA is then transformed into electroporation-competent E. coli XL1-Blue. Several frozen stocks are made from cultures of the mutant libraries and stored at −80° C. for future use. The complexity of the library (over 10⁷ unique clones) is determined by counting the number of colonies obtained after plating an aliquot of the freshly transformed cells on agar-containing LB medium and ampicillin. Presence of the expected mutations is verified by DNA sequencing of randomly picked clones.

### Example 10

### Selection of hCASK PDZ Variant Recognizing Bacillus anthracis Protein BclA

Affinity Selection

In this example, the error-prone PCR library of example 6, above, is selected for mutants that are capable of recognizing a peptide of the sequence SASIIIEKVA (SEQ ID NO: 26) corresponding to the C-terminus of protein BclA which is found in the exosporium of Bacillus anthracis spores. Phage displaying the hCASK-PDZ library variants are prepared according to standard methods (e.g., Barbas et al., 2001) from frozen stocks of the library. The library is carried through 5 rounds of panning using N-terminal-biotinlylated peptide SASIIIEKVA bound to streptavidin coated onto polystyrene wells of multiwell plates (Nunc). Phage binding specifically to SASIIIEKVA peptide-coated wells are allowed to infect E. coli XL1-Blue simply by adding cells to the well and incubating them for 15 minutes. The input phage titer (number of phage added to a well) and output phage (phage removed from well) from each round are determined. The ratio of output phage to input phage for each round of panning typically shows a clear trend of phage amplification after Round 3 or 4, suggesting selection of mutants specific for the target peptide SASIIIEKVA.

Mutant Screening

A phage ELISA is performed on about 20 randomly chosen clones from each of panning rounds 3, 4 and 5 to verify that the selection is successfully amplifying mutants binding specifically to peptide SASIIIEKVA. Log-phase XL1-Blue cultures are infected with the mutant phage output from each panning round. An aliquot of this infected culture is then plated onto agar-containing medium and allowed to grow 16 hours. Multiple clones are picked, grown in 96-well polypropylene culture plates, infected with helper phage, and the resulting culture supernatant used in a phage ELISA. Several clones from round 5 produce a strong binding signal to peptide SASIIIEKVA and are chosen for further characterization.

Mutant Characterization

Phage are purified by PEG/NaCl precipitation from three mutants as well as wildtype controls and tested against biotinylated peptides HRRSARYLDTVL (SEQ ID NO: 27), QKAPTKEFYA (SEQ ID NO: 13), and SASIIIEKVA by phage ELISA. Each mutant shows dramatically improved ELISA signal for peptide SASIIIEKVA compared to wildtype hCASK-PDZ, and only weak binding to control peptides HRRSARYLDTVL and QKAPTKEFYA. These mutants are therefore capable of specifically binding to peptide SASIIIEKVA.

Confirmation of BclA Binding

Characterized mutants showing binding to peptide SASIIIEKVA are then further characterized; their ability to bind Bacillus anthracis BclA is determined. Phagemid DNA of the selected mutants is purified per standard methods, digested with EcoRI and BamHI and the resulting variant hCASK-PDZ gene fragment is ligated to pGEX-2TK DNA digested with the same restriction enzymes. The resulting ligated DNA is transformed into E. coli strain JM109 to yield clones containing plasmid pGEX-PDZ-variant. These clones are grown to an OD600 of 0.5 to 1.0 and induced using 1 mM IPTG for 5 hours at 22° C. The induced cells are pelleted by centrifugation and lysed using a French press. PDZ variant-GST fusion protein is purified from the lysate via affinity chromatography using glutathione sepharose affinity medium. Purified PDZ variant-GST fusion protein is then tested for its ability to bind Bacillus anthracis BclA by using the PDZ variant-GST fusion as an affinity reagent, as described in example 5.1 above, wherein protein BclA is present on a western blot or coated onto the wells of a multi-well plate in an ELISA format.

### Example 11

### Recursive Ensemble Mutagenesis of hCASK PDZ Gene

The random combinatorial library described in example 7 is subjected to affinity selection, as described in example 10. A few different PDZ variants capable of binding target peptide SASIIIEKVA are isolated. The aim of the present example is to isolate further variants having improved binding affinity towards the target peptide. The DNA sequences of the few different PDZ variants are determined and used to design a new combinatorial library wherein a bias is introduced towards the expression of those amino acids observed at the randomized positions of the isolated PDZ variants (Delagrave et al., 1993, Protein Eng, 6: 327-31). To design this new library, the amino acids encountered in the isolated PDZ variants are compiled for each mutated position (i.e., residues M501, I503, L505, Q553, L556 and R557). The list of amino acids is entered into a computer program called CyberDope, available from Kairos Scientific (San Diego, Calif.). The program is instructed by the operator to use the group probability option (PG) and the NNK (NN[G/T]) codon option. The program then provides a nucleotide mixture (degenerate codon) which encodes all of the amino acids encountered at the mutated position of interest. A list of amino acids is entered for each mutated position (i.e., residues M501, I503, L505, Q553, L556 and R557) resulting in a degenerate codon for each position.

Oligonucleotides comprising the degenerate codons provided by the computer program are synthesized by a custom oligonucleotide manufacturer (Integrated DNA Technologies, Coralville, Iowa). Using these oligos, a new combinatorial library is synthesized by PCR. The new library is then selected by affinity panning, as described in example 10. New variants having improved affinity are selected from this library. The selected further PDZ variants are then tested for improved ability to bind the target protein, as described in example 13, using BIAcore measurements of affinity.

### Example 12

### Affinity Maturation of Wildtype PDZ

The affinity of the wildtype PDZ of hCASK towards its target protein syndecan-2 can be improved by a process of in vitro affinity maturation. This is done by first mutating, via error-prone PCR, the gene of hCASK PDZ, thereby creating a library as described in example 6. Secondly, affinity selection is applied to select for variants having improved affinity as described in example 10, except that biotinylated peptide QKAPTKEFYA, corresponding to the C-terminus of syndecan-2, is used instead of biotinylated peptide SASIIIEKVA. Individual selected variants are grown, their DNA purified, and the PDZ gene fragments of the variants are sub-cloned, as described in examples 2 and 10. The resulting GST-PDZ variant fusions are purified, per example 10, and tested to compare their affinities towards the target protein syndecan-2. The cytoplasmic domain of syndecan-2 is attached to the microfluidic chip of a BIAcore instrument (BIAcore, Piscataway, N.J.) and GST-PDZ variants are analyzed using the instrument to calculate their binding affinities. Variants showing improved affinity compared to the parent hCASK PDZ demonstrate the effectiveness of this procedure. Such improved variants can be useful as research reagents, diagnostics or therapeutics.

### Example 13

### Affinity Maturation of PDZ Variants

The affinity of a PDZ variant isolated in example 10 above, towards its target protein, BclA, can be improved by a process of in vitro affinity maturation. This is done by first mutating, via error-prone PCR, the gene of the PDZ variant, thereby creating a library as described in example 6. Secondly, affinity selection is applied to select for variants having improved affinity, as described in example 10. Individual variants are grown, their DNA purified, and the PDZ gene fragments of the variants are sub-cloned, as described in examples 2 and 10. The resulting GST-PDZ variant fusions are purified, per example 10, and tested to compare their affinities towards the target protein. The target protein is attached to the microfluidic chip of a BIAcore instrument (BIAcore, Piscataway, N.J.) and GST-PDZ variants are analyzed using the instrument, to calculate their binding affinities. Variants showing improved affinity compared to the parent PDZ variant demonstrate the effectiveness of this procedure. Such improved variants can be useful as research reagents, diagnostics or therapeutics.

### Example 14

### Construction of PDZ Variant Fusion Proteins and their Use as Affinity Reagents

Any of the evolved PDZ variants isolated in examples 13, 12, 11, or 10 can be made into translational fusions essentially as described for wildtype PDZ domain in examples 2, 3 and 4. Any of the resulting fusion proteins can be used as reagents for detection of the peptides or proteins which these PDZ variants have been evolved to bind, essentially as described in examples 5.1 and 5.2.

### Example 15

### Affinity Purification Using Evolved PDZ Domain

An evolved PDZ domain binding to a target protein is isolated according to any of the above examples. The evolved PDZ domain is purified and attached to beaded agarose affinity medium using the Aminolink Plus immobilization kit (Pierce, www.piercenet.com). The target protein is then isolated from a complex mixture by using the immobilized evolved PDZ domain according to standard affinity chromatography procedures.

### Example 16

### Sequences

SEQ ID NO: 1. DNA sequence of hCASK-PDZopt.
TTTTTATGCGGCCCAGCCGGCCGGATCCGGTATGGATATGGAAAATGTTA
CCCGTGTTCGTTTAGTTCAATTTCAAAAAAATACCGATGAACCAATGGGT
ATTACCTTGAAAATGAATGAATTGAATCATTGTATTGTTGCCCGTATTAT
GCATGGTGGTATGATTCATCGTCAAGGTACTTTGCATGTTGGTGATGAAA
TTCGTGAAATTAATGGTATTTCTGTTGCCAATCAAACCGTTGAACAATTG
CAAAAAATGTTGCGTGAAATGCGTGGTTCTATTACCTTTAAAATTGTTCC
ATCTTATCGTACCCAATCTTCTTCTGGAATTCATGCGGCCGCTGGTGCTC
CAGT
SEQ ID NO: 2. Amino acid sequence encoded by the
underlined DNA sequence of hCASK-PDZopt gene
fragment shown in SEQ ID NO: 1.
GMDMENVTRVRLVQFQKNTDEPMGITLKMNELNHCIVARIMHGGMIHRQG
TLHVGDEIREINGISVANQTVEQLQKMLREMRGSITFKIVPSYRTQSSS
SEQ ID NO: 3. Amino acid sequence of hCASK-PDZ-GST
fusion.
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSRRASVGSGMDMENVTRVRLVQFQK
NTDEPMGITLKMNELNHCIVARIMHGGMIHRQGTLHVGDEIREINGISVA
NQTVEQLQKMLREMRGSITFKIVPSYRTQSSSGIHRD

(The hCASK-PDZ amino acid sequence is shown in italics.)

SEQ ID NO: 4. Amino acid sequence of hCASK-PDZ-
alkaline phosphatase fusion.
MSIQHFRVALIPFFAAFCLPVFAGMDMENVTRVRLVQFQKNTDEPMGITL
KMNELNHCIVARIMHGGMIIHRQGTLHVGDEIREINGISVANQTVEQLQK
MLREMRGSITFKIVPSYRTQSSSRTPEMPLQGTAVDGGGGSMHASLEVLE
NRAAQGDITAPGGARRLTGDQTAALRDSLSDKPAKNIILLIGDGMGDSEI
TAARNYAEGAGGFFKGIDALPLTGQYTHYALNKKTGKPDYVTDSAASATA
WSTGVKTYNGALGVDIHEKDHPTILEMAKAAGLATGNVSTAELQDATPAA
LVAHVTSRKCYGPSATSEKCPGNALEKGGKGSITEQLLNARADVTLGGGA
KTFAETATAGEWQGKTLREQAQARGYQLVSDAASLNSVTEANQQKPLLGL
FADGNMPVRWLGPKATYHGNIDKPAVTCTPNPQRNDSVPTLAQMTDKAIE
LLSKNEKGFFLQVEGASIDKQDHAANPCGQIGETVDLDEAVQRALEFAKK
EGNTLVIVTADHAHASQIVAPDTKAPGLTQALNTKDGAVMVMSYGNSEED
SQEHTGSQLRIAAYGPHAANVVGLTDQTDLFYTMKAALGLK

(the hCASK-PDZ domain sequence is italicized, the leader sequence (E. coli β-lactamase TEM) is underlined, and the remainder of the sequence corresponds to alkaline phosphatase of E. coli.)

SEQ ID NO: 5. Amino acid sequence of hCASK-PDZ-Fc
fusion protein.
MRAPAQIFGFLLLLFPGTRCGMDMENVTRVRLVQFQKNTDEPMGITLKMN
ELNHCIVARIMHGGMIHRQGTLHVGDEIREINGISVANQTVEQLQKMLRE
MRGSITFKIVPSYRTQSSSEPKSCDKTHTCPPCPAPELLGGPSVFLFPPK
PKDTLMISRTPEVTCVVVDVSHEDPEVKFNWYVDGVEVHNAKFKPREEQY
NSTYRVVSVLTVLHQDWLNGKDYKCKVSNKALPAPMQKTISKAKGQPREP
QVYTLPPSRDELTKNQVSLTCLVKGFYPRHIAVEWESNGQPENNYKTTPP
VLDSDGSFFLYSKLTVDKSRWQQGNVFSCSVMHEALHNHYTQKSLSLSPG
K

(Underlined sequence is the signal sequence of light chain human immunoglobulin. Italicized sequence is the hCASK-PDZ domain, and the remainder of the sequence corresponds to the human IgG1 Fc domain sequence.)

SEQ ID NO: 6. Amino acid sequence of polyhistidine
tagged and secreted hCASK-PDZ.
MSIQHFRVALIPFFAAFCLPVFAGMDMENVTRVRLVQFQKNTDEPMGITL
KMNELNHCIVARIMHGGMIHRQGTLHVGDEIREINGISVANQTVEQLQKM
LREMRGSITFKIVPSYRTQSSSHHHHHH

(The hCASK-PDZ domain sequence is italicized, the leader sequence (E. coli β-lactamase TEM) is underlined, and the C-terminal six residues correspond to the polyhistidine tag.)

SEQ ID NO: 7. Amino acid sequence of secreted
hCASK-PDZ.
MSIQHFRVALIPFFAAFCLPVFAGMDMENVTRVRLVQFQKNTDEPMGITL
KMNELNHCIVARIMHGGMIHRQGTLHVGDEIREINGISVANQTVEQLQKM
LREMRGSITFKIVPSYRTQSSS

(The hCASK-PDZ domain sequence is italicized, the leader sequence (E. coli β-lactamase TEM) is underlined.)

SEQ ID NO: 8. human NHERF PDZ dimer
PRLCCLEKGPNGYGFHLHGEKGKLGQYIRLVEPGSPAEKAGLLAGDRLVE
VNGENVEKETHQQVVSRIRAALNAVRLLVVDPETDEQLQKLGVQVREELL
RAQEAPGQAEPPAAAEVQGAGNENEPREADKSHPEQRELRPRLCTMKKGP
SGYGFNLHSDKSKPGQFIRSVDPDSPAEASGLRAQDRIVEVNGVCMEGKQ
HGDVVSAIRAGGDETKLLVVDRETDEFFKKCRVI

(Human NHERF protein fragment comprising two PDZ domains.)

SEQ ID NO: 9. Amino acid sequence of the third PDZ domain of human Dlg1 (NOT IN ITALICS) fused to a signal sequence and gene 3 coat protein (ITALICS) as provided in the pCANTAB5E phage display vector:

MKKLLFAIPLVVPFYAAQPAAVLGDDEITREPRKVVLHRGSTGLGFNIVG
GEDGEGIFISFILAGGPADLSGELRKGDRIISVNSVDLRAASHEQAAAAL
KNAGQAVTIVAQYRPEEYSRFEAAAAGAPVPYPDPLEPRAAQTVESCLAK
PHTENSFTNVWKDDKTLDRYANYEGCLWNATGVVVCTGDETQCYGTWVPI
GLAIPENEGGGSEGGGSEGGGSEGGGTKPPEYGDTPIPGYTYINPLDGTY
PPGTEQNPANPNPSLEESQPLNTFMFQNNRFRNRQGALTVYTGTVTQGTD
PVKTYYQYTPVSSKAMYDAYWNGKFRDCAFHSGFNEDPFVCEYQGQSSDL
PQPPVNAGGGSGGGSGGGSEGGGSEGGGSEGGGSEGGGSGGGSGSGDFDY
EKMANANKGAMTENADENALQSDAKGKLDSVATDYGAAIDGFIGDVSGLA
NGNGATGDFAGSNSQMAQVGDGDNSPLMNNFRQYLPSLPQSVECRPFVFS
AGKPYEFSIDCDKINLFRGVFAFLLYVATFMYVFSTFANILRNKES.

SEQ ID NO: 10. Nucleotide sequence of the third PDZ domain of human Dlg1 cloned into the pCANTAB5E phage display vector, between SfiI and NotI restriction sites (in italics):

TTATTATTCGCAATTCCTTTAGTTGTTCCTTTCTATGCGGCCCAGCCGGC
CGCAGTACTTGGAGATGATGAAATTACAAGGGAACCTAGAAAAGTTGTTC
TTCATCGTGGCTCAACGGGCCTTGGTTTCAACATTGTAGGAGGAGAAGAT
GGAGAAGGAATATTTATTTCCTTTATCTTAGCCGGAGGACCTGCTGATCT
AAGTGGAGAGCTCAGAAAAGGAGATCGTATTATATCGGTAAACAGTGTTG
ACCTCAGAGCTGCTAGTCATGAGCAGGCAGCAGCTGCATTGAAAAATGCT
GGCCAGGCTGTCACAATTGTTGCACAATATCGACCTGAAGAATACAGTCG
TTTTGAAGCTGCGGCCGCAGGTGCGCCGGTGCCGTATCCGGATCCGCTGG
AACCGCGTGCCGCATAGACTGTTGAAAGTTGTTTAGCAAAACCTCATACA
GAAAATTCATTTACTAACGTCTGGAAAGACGACAA.

### Example 17

### Iterative Evolution to Achieve High-Affinity PDZ Domains

The affinity-matured variants of examples 12 and 13 can be further mutated and selected to achieve additional improvements in affinity. This is done by simply iterating the process described in examples 12 and 13, with the option of omitting detailed affinity characterization between each round of mutagenesis and selection. Thus, the gene encoding an affinity-matured variant isolated in examples 12 or 13 is mutated by error-prone PCR as described above, and the resulting population of mutant genes is cloned in a phage display vector to yield a phage display library. The library of variants is selected for variants having superior affinity to the target. The selected variants are optionally characterized or further mutated to create a further phage display library which is selected for further variants having superior affinity. Evolved PDZ domains having affinities (dissociation constant, or K_{d}) for their target of 100 nM, 10 nM, 1 nM or better.

### Example 18

### Directed Evolution of PDZ Dimer

A polynucleotide encoding the polypeptide of SEQ ID NO: 8, comprising two PDZ domains, is mutated by error-prone PCR in substantially the same way as hCASK PDZ in Example 6, except that primers specific to the 5′ and 3′ ends of the polynucleotide are used. The mutated PCR product is cloned, substantially as described above, in a phage display vector and a library of phage displaying variants of the polypeptide of SEQ ID NO: 8 is produced. This library is subjected to affinity panning with a single target peptide, and variants binding specifically to the target are isolated. The resulting PDZ dimer variant binds target peptide (or proteins having the same C-terminal sequence) with greater avidity than monomeric PDZ domains.

### Example 19

### Directed Evolution Using a Protein Target Instead of a Peptide

In example 10, a peptide corresponding to the C-terminal residues of BclA is used to select PDZ variants binding to BclA. A different approach is to use protein BclA itself as the target. The protein target is immobilized by adsorption to a well of a polystyrene microtiter plate, as is routinely done to carry out ELISAs. Alternatively, antibodies specific to BclA are adsorbed to the microtiter plate and used to bind specifically to the target protein BclA. Affinity selection is carried out and variants binding to the target protein are selected. Care is taken to avoid selection of PDZ variants binding to anti-BclA antibodies, if they are used to immobilize BclA, by pre-binding the phage display library to immobilized antibodies in the absence of BclA.

### Example 20

### Phage Display of the Third PDZ Domain of Human Dlg1

As described in Example 6 above, the third PDZ domain of human protein Dlg1 was phage displayed and mutated by error-prone PCR in preparation for affinity selection of novel variants. The sequence of Dlg1 PDZ3 cloned in vector pCANTAB5E was confirmed by DNA sequencing. The complexity of the error-prone PCR library was determined to be 2.2×10⁶ transformants. The mutation rate was found to be 2.1 nucleotide mutations per clone. The ability of the phage-displayed PDZ domain to bind its ligand (N-terminal biotinylated peptide having the sequence “SSLQSLETSV”) specifically was shown by phage ELISA as described in Example 6.

### Example 21

### An Evolved Dlg1 PDZ3 Variant Having a Stronger Phage ELISA Signal

The library of Example 20 was panned as described in above Example 10, with the added specification that phage growth after helper phage superinfection was carried Out at 30° C. instead of 37° C. One clone, named B2, was isolated showing increased phage ELISA signal for all peptides against which it was tested while preserving its preference for peptide SSLQSLETSV (see Table 2 below). The data can be interpreted that Variant B2 can be a superior parent PDZ clone to carry out affinity selection on novel ligand targets because of its greater ease of screening and characterization by phage ELISA.

TABLE 2
Phage ELISA showing OD405
after 35 minutes of incubation at room temperature.

Phage tested

| Ligand | Dlg1-PDZ3 | B2 |
|---|---|---|
| dtvl | 0.510 | 1.168 |
| etsv | 0.849 | 1.606 |
| efya | 0.460 | 1.153 |
| ekva | 0.496 | 1.133 |

Ligand sequences of Table 2 include: dtvl: biotin-HRRSARYLDTVL; etsv: biotin-SSLQSLETSV (SEQ ID NO: 28); efya: biotin-QKAPTKEFYA; ekva: biotin-SASIIIEKVA.

Briefly, ELISA was carried out by immobilizing 1 g streptavidin (Jackson Labs) into wells of microtiter plates, binding 1 μg of biotinylated peptides to the streptavidin-coated wells, blocking wells with I-BLOCK (Tropix, Bedford Mass.), adding 100 μL PEG-precipitated phage pellets resuspended in 1×PBS, detected with anti-M13 horseradish-peroxidase-labeled antibody (Amersham) and ABTS (Sigma), according to instructions provided in Recombinant Phage Antibody System (Amersham).

### Example 22

### Selection of PDZ Variants Binding to the Light Chain of Botulinum Neurotoxin

Step 1. Creation of a Library of PDZ Domain Variants.

A library of PDZ domain variants are prepared by amplifying a PDZ gene using error-prone PCR and cloning the mutated gene in a phage display vector. The resulting transformants (library size of 1 to 10 million clones) are infected with helper phage to yield a library of variants displayed oil bacteriophage.

In preparation for the proposed project, a gene fragment encoding the third PDZ domain of human protein Discs Large Homolog 1 (Dlg1) is cloned and Mutated as described in Example 20. Additional libraries of the same gene but with higher mutation rates (e.g., 5 amino acid Substitutions per mutant, or 10 Substitutions per mutant) can produced by increasing the MgCl₂ concentration in the error-prone PCR reaction, decreasing the amount of template or increasing the number of amplification cycles.

Phage displaying the Dlg1-PDZ3 library variants are prepared according to standard methods from frozen stocks of the library by superinfecting the transformed cells with helper phage. After overnight Culture at 30° C., the cells are removed from the Culture medium by centrifugation and the phage are isolated by PEG/NaCl precipitation.

Step 2. Affinity Selection of PDZ Variants Binding to BoNT-Lc, and Preliminary Characterization.

The light chains of botulinum neurotoxin types A and B are immobilized onto solid supports and used to isolate phage displaying PDZ domain variants capable of binding to these targets—a process called ‘affinity selection’ or ‘panning’. After a few rounds of panning, individual phage clones are isolated and their DNA extracted to determine the sequences of the PDZ variants they encode. Their affinity for the light chains are estimated via phage ELISA.

Affinity Selection

The error-prone PCR library discussed above is selected for mutants that are capable of recognizing the light chains of BoNT/A and BoNT/B. The light chains are purchased from List Biological Laboratories. Five separate aliquots of the phage library are carried through 5 rounds of panning using the BoNT-Lc coated onto polystyrene wells of multiwell plates (Nunc). After extensive washing of the wells with PBST, phage binding specifically to Lc-coated wells are allowed to infect E. coli DH5αFT simply by adding these cells to the well and incubating them for 60 minutes at 37° C. The input phage titer (number of phage added to a well) and output phage titer (specifically bound phage removed from well) from each round are determined. The ratio of output phage to input phage for each round of panning typically shows a clear trend of phage amplification after Round 3 or 4, suggesting selection of mutants specific for the target Lc. A positive control peptide, SSLQSLETSV is expected to be recognized by and to select for wildtype Dlg1 PDZ3 and any variants of improved affinity for this peptide.

Mutant Screening

For each Lc, a phage ELISA is performed on about 20 randomly chosen clones from each of panning rounds 3, 4 and 5 to verify that the selection is successfully amplifying mutants binding specifically to the target molecules. (20 clones×3 rounds×2 BoNT-Lc=120 ELISA wells=only two 96-well plates.) Log-phase DH5αFT cultures are infected with an aliquot of the mutant phage output from each panning round. An aliquot of this infected culture is then plated onto agar-containing medium and phagemid-transformed colonies are allowed to grow 16-24 hours. Multiple clones are picked, grown in 96-well polypropylene culture plates, infected with helper phage, and the resulting culture supernatants tested in a phage ELISA. Several clones from round 5 should produce a strong binding signal to the appropriate targets (i.e., round 5 clones selected for binding to BoNT/A-Lc should bind well to this protein and poorly to BoNT/B-Lc, and vice versa). These PDZ variants are then chosen for further characterization.

Mutant Characterization

Phage clones (˜3 per Lc) showing specific binding to target BoNT-Lc in the primary screen are grown in culture volumes of 25 mL, purified by PEG/NaCl precipitation and tested again by phage ELISA against Lc. Controls, including wildtype Dlg1 PDZ3 phage are included as well. Mutants showing strong and reproducible ELISA signal for their respective target Lc are archived (frozen stocks) and sequenced.

To estimate PDZ variant affinity for its target Lc, a competition ELISA is carried out wherein a constant amount of phage is incubated with serial dilutions of soluble (non-immobilized) competing Lc. A plot of absorbance (ELISA signal) vs. soluble protein concentration shows a sigmoidal curve, the inflection point of which provides an estimate of K_{d} (dissociation constant). Phagemid DNA is isolated from separate cultures of the clones showing high apparent affinity. This DNA is sequenced according to standard methods to determine the predicted amino acid sequence of the selected PDZ variants. Computer program suites such as DNASTAR are used to analyze sequence data files (electropherograms) and align multiple predicted protein sequences, thereby facilitating analysis.

Step 3. Purification and Characterization of PDZ Variants.

The genes of PDZ variant clones of the highest apparent affinity are sub-cloned into an expression vector for protein purification and further characterization. An expression vector comprising a signal sequence and a poly-histidine affinity-purification tag is used for expression of the PDZ variant in E. coli. The resulting protein is purified through its poly-histidine tag and characterized. Characterization parameters include: affinity for BoNT-Lc, specificity, stability, and ability to decrease Lc enzymatic activity in vitro.

Subcloning and Purification

Mutants showing reproducible binding to Lc are then further characterized; the affinity of isolated PDZ variants to BoNT-Lc protein is determined. To do this, mutant DNA is first subcloned into an expression vector providing a polyhistidine affinity tag at the C-terminus. Phagemid DNA of the selected mutants is purified per standard methods, and the PDZ variant ORF is amplified by high-fidelity PCR using appropriate primers. The resulting PCR product is digested with EcoRI and HindIII and ligated to pQE-70 DNA (Qiagen) digested with the same restriction enzymes. The resulting ligated DNA is transformed into E. coli strain DH5αFT to yield clones containing plasmid pQE-70-PDZ-variant. These clones are grown to an OD600 of 0.5 to 1.0 and induced using 0.1 to 1 mM IPTG for 5 hours at 30° C. The induced cells are pelleted by centrifugation and lysed using a French press. PDZ variant is purified from the clarified lysate via immobilized metal ion affinity chromatography (IMAC) Talon resin (BD Biosciences). This process is carried out essentially in parallel for all the selected mutants (−2 to 6 clones).

Affinity Measurement

An ELISA is performed to rapidly estimate binding affinity of purified PDZ variants to BoNT-Lc. Lc is immobilized, as described above, by adsorption onto polystyrene microtiter plates. Various dilutions of PDZ variants are incubated with the immobilized ligand and unbound protein is washed away using PBST. PDZ proteins that remain bound are detected using peroxidase-labeled anti-His₆ tag antibody (Roche Applied Science). Colorimetric reporter ABTS is added to the wells and the color change is read using a plate reader.

Affinity of purified PDZ protein to BoNT-Lc is more quantitatively measured by surface plasmon resonance implemented on a BIAcore instrument (Biacore). BoNT-Lc is immobilized on a microfluidic chip and PDZ variants in solution are allowed to bind to the immobilized toxin subunit. Information concerning binding kinetics is collected and analyzed to yield on and off rates as well as affinity constants. Each PDZ variant will be tested against its selected toxin subunit.

Binding affinities can be obtained either from rate constant measurements (the dissociation constant K_{d} is the ratio of the rate constants k_{d}/kₐ for a 1:1 interaction) or by measuring the steady state level of binding as a function of sample concentration.

Neutralization of Lc Protease Activity In Vitro

PDZ variants having good binding affinity for BoNT-Lc are tested for their ability to decrease Lc enzymatic activity in vitro. BoNT-Lc (obtained from List Biologicals) is incubated in the presence of decreasing concentrations of PDZ variant and of a constant concentration of substrate SNAPtide™ (FITC/DABCYL) (List Biological Laboratories) in 20 mM HEPES, pH 8.0 containing 0.3 mM ZnCl₂, 1.25 mM dithiothreitol (DTT) and 0.1% Tween 20. SNAPtide™ substrate fluorescence is internally quenched via FRET, but hydrolysis of the substrate releases a fluorescein-labeled cleavage product which is readily detected. Hydrolysis of the SNAPtide™ substrate by BoNT/A-Lc is easily followed by exposing the sample to fluorescence excitation (λ_{excitation}=490 nm) and monitoring fluorescence emission (λₑₘᵢₛₛᵢₒₙ=523 nm) using a fluorescence-capable plate reader (e.g., VICTOR³™ Multilabel Counter; Perkin Elmer).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A polypeptide comprising an engineered PDZ domain, wherein said engineered PDZ domain binds to a target associated with a pathogen or disease state.

2. The polypeptide of claim 1 wherein said pathogen is viral, fungal, or bacterial.

3. The polypeptide of claim 1 wherein said pathogen is Bacillus anthracis or Clostridium botulinum.

4. The polypeptide of claim 1 wherein said target is protein BclA of Bacillus anthracis or a fragment thereof.

5. The polypeptide of claim 1 wherein said target is a polypeptide having a C-terminal sequence of EFYA.

6. The polypeptide of claim 1 wherein said PDZ domain is evolved.

7. The polypeptide of claim 6 wherein said polypeptide binds to said target with a dissociation constant (K_{d}) of about 100 nM or lower.

8. The polypeptide of claim 7 wherein said polypeptide binds to said target with a dissociation constant of about 15 nM or lower.

9. The polypeptide of claim 1 further comprising a reporter group.

10. The polypeptide of claim 1 further comprising an effector domain.

11. The polypeptide of claim 1 further comprising a radioactive isotope.

12. The polypeptide of claim 1 wherein said polypeptide is isolated.

13. A polynucleotide encoding the polypeptide of claim 1.

14. A vector comprising the polynucleotide of claim 12.

15. A host cell comprising the polynucleotide of claim 12.

16. An isolated antibody that binds to said polypeptide of claim 1.

17. A method of detecting the presence of a pathogen or disease in a patient comprising:
a) administering a polypeptide of claim 1 to said patient; and
b) detecting binding of said polypeptide in said patient.

18. A method of detecting the presence of a pathogen or disease in a sample comprising:
a) contacting a polypeptide of claim 1 with said sample; and
b) detecting binding of said polypeptide to said sample.

19. A method of preparing a polypeptide comprising a PDZ domain, wherein said PDZ domain binds to a target produced by a pathogen or disease state, comprising:
a) creating a library of polypeptides from one or more parent polypeptides comprising a PDZ domain;
b) identifying one or more polypeptides from said library having binding affinity for said target.

20. A method of treating a disease, comprising administering to a patient afflicted with or likely to be afflicted with said disease a therapeutically effective amount of a polypeptide comprising a PDZ domain capable of binding to a target associated with said disease.

21. The method of claim 20 wherein said disease is associated with a pathogen.

22. The method of claim 21 wherein said pathogen is Bacillus anthracis, Clostridium botulinum or Clostridium tetani.

23. A method of preparing a polypeptide comprising a PDZ domain, wherein said PDZ domain binds to a polypeptide target associated with a pathogen, comprising:
a) forming a library of polypeptides from one or more parent polypeptides comprising a PDZ domain;
b) selecting a first polypeptide from said library, said first polypeptide having binding affinity to an intermediate target having 20% to 80% sequence identity in the last 5 amino acids with the last 5 amino acids of said target;
c) creating a further library of polypeptides from the first polypeptide of step b);
d) repeating steps b) and c) until a polypeptide that binds with said target is identified.
